(19) United States
(12) Patent Application Publication
(10) Pub. No.: US 2006/0293249 A1
(43) Pub. Date: Dec. 28, 2006

(54) Porphyrin derivatives

(75) Inventors: Gokhan Yahioglu, London (GB); Delisa Ibanez Garcia, Puerto Sagunto (ES)

Correspondence Address: FISH & RICHARDSON PC, P.O. BOX 1022, MINNEAPOLIS, MN, 55440-1022, US

(73) Assignee: PhotoBiotics Limited

(21) Appl. No.: 11/134,955

(22) Filed: May 23, 2005

Related U.S. Application Data
(63) Continuation of application No. PCT/GB03/05128, filed on Nov. 21, 2003.

(30) Foreign Application Priority Data
Nov. 21, 2002 (GB) GB20020027259

(51) Int. Cl. A61K 31/7056; A61K 31/555; A61K 31/69; A61K 31/409; A61K 31/444; C07D 487/22; C07H 15/02

(52) U.S. Cl. 514/23; 514/185; 514/332; 514/64; 514/410; 536/17.4; 540/145

(57) ABSTRACT

A compound of formula I is disclosed: 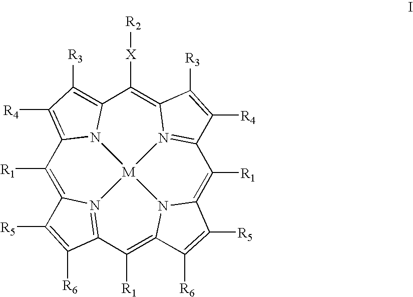
wherein each R₁ is independently 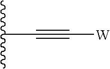 wherein W is an aryl, alkyl or heteroaryl group, each of which may be independently and optionally substituted by one or more of:
   OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, or a sugar derivative;
R₂ is H, a halogen, an isothiocyanate group, a haloacetamide, maleimide, Y-aryl or Y-heteroaryl, where Y is O, S, NH, C(O) or CO₂, and where said aryl or heteroaryl group may be optionally substituted by one or more of:
   OH, halogen, an isothiocyanate group, a haloacetamide, maleimide, COOH, NO₂, NH₂, alkyl, haloalkyl, alkoxy, (CO)_{n′}(O)_{m′}Z′, a polyethylene glycol group, an alkyl sulfonate group, an alkyl-COOH group, a substituted or unsubstituted benzyl group, or a sugar derivative; Z and Z′ are each independently silicon-containing protecting groups and m, m′, n and n′ are each independently 0 or 1;
X is a C₁₋₂₀ alkylene group, optionally substituted by one or more substituents selected from halogen, NO₂, CN, OH, OMe, NH₂, CF₃, COOH and CONH₂;
each R₃, R₄, R₅ and R₆ is independently H, alkyl, alkoxy, halogen or OH; and
M is 2H or a metal.

### RELATED APPLICATIONS

This application is a continuation of international application no. PCT/GB2003/005128 filed 21 Nov. 2003 and claims priority to British Application no. GB20020027259 filed 21 Nov. 2002, both of which applications are incorporated herein by reference.

### FIELD OF THE INVENTION

The present invention relates to porphyrin derivatives and related pharmaceutical compositions and methods.

### BACKGROUND

Porphyrins have found uses in numerous applications including precursors for novel conducting polymers [Wagner et al, J. Am. Chem. Soc., 1994, 116, 9759; Anderson, Inorg. Chem., 1994, 33, 972 and Arnold et al, Tetrahedron, 1992, 48, 8781]; non-linear optically active (NLO) materials [Anderson et al, Angew. Chem. Int. Ed. Engl., 1994, 33, 655 and Arnold et al, J. Am. Chem. Soc., 1993, 115, 12197]; photosynthetic model compounds [Wagner et al, J. Org. Chem., 1995, 60, 5266, and Lin et al, Science, 1994, 264, 1105]; and enzyme mimics [Anderson et al, Angew. Chem. Int. Ed. Engl., 1990, 29, 1400; Anderson et al, J. Chem. Soc., Chem. Commun., 1992, 946 and Mackay et al, J. Chem. Soc., Chem. Commun., 1992, 43]. Meso-tetraalkynyl-substituted porphyrins are reported by Anderson in Tetrahedron Lett., 1992, 33 1101. Porphyrins have also been the focus of investigations in the field of photodynamic therapy.

Photodynamic therapy (PDT) is a promising new medical treatment that involves the combination of visible light, a drug (photosensitiser) and oxygen to bring about a cytotoxic effect to cancerous or otherwise unwanted tissue. The photosensitiser absorbs light of the appropriate wavelength and undergoes one or more electronic transitions emerging in its excited triplet state. The excited photosensitiser can participate in a one-electron oxidation-reduction reaction (termed Type I) with a neighbouring molecule, producing free radical intermediates that can react with oxygen to produce peroxy radicals and various reactive oxygen species (ROS). Alternatively, the triplet-state photosensitiser can transfer its energy to molecular oxygen (termed Type II) generating singlet molecular oxygen, a highly reactive, powerful and indiscriminate oxidiser that readily reacts with a variety of biological molecules and assemblies. It is generally accepted that singlet oxygen is the primary cytotoxic agent in PDT.

The first PDT photosensitiser to win approval by the regulatory authorities was Photofrin, a complex mixture of the more active porphyrin oligomers that comprise haematoporphyrin derivative (HpD). This is derived from haematoporphyrin by reaction with acetic and sulphuric acids, and commercially available Photofrin is a purified version of this mixture. PDT using Photofrin has been approved for the treatment of lung and oesophagel cancer in the US and for several other cancers worldwide. However, Photofrin suffers from a number of limitations. Firstly, it is in the form of a complex mixture which makes it difficult to ascertain precisely how the drug works and how it interacts with tissues in the body. Secondly, Photofrin has a tendency to be retained in skin for five to six weeks, inducing undesirable and prolonged photosensitivity under normal daylight. Finally, the longest wavelength of light at which the drug can be photoactivated (630 nm) is well below the wavelength needed for maximum tissue penetration. These limitations have led to the synthesis and development of a range of second and third generation photosensitisers.

Ideally, photosensitisers for PDT should
(i) be chemically pure and of known (constant) composition;
(ii) have minimal dark toxicity and only be cytotoxic in the presence of light;
(iii) have a strong absorption at longer wavelengths (between 650-800 nm) when tissue penetration of light is at a maximum, while still being energetic enough to produce singlet oxygen;
(iv) exhibit a high quantum yield for singlet oxygen;
(v) be rapidly excreted from the body, thereby inducing low systemic toxicity;
(vi) be preferentially retained by the target tissue;
(vii) be water soluble and easy to formulate (to aid delivery of the drug), and stable to avoid the formation of metabolites.

Porphyrins usually have a characteristic red colour due to an intense absorption (called the B band: a pi-pi* transition) in the region 400-420 nm. In the visible part of the spectrum, there are weaker absorptions (called Q bands) in the region 500-650 nm, the number depending on whether the macrocycle is in the form of a free-base (M=2H, 4 bands), a dication (M=4H, 2 bands) or a metal complex (M=M, 2 bands).

Porphyrins absorb strongly in the blue, which means they are not well “tuned” to work with red light and so their PDT effect is not very penetrating. Thus, macrocycles with intense, longer-wave absorptions (e.g., chlorins, phthalocyanines, benzporphyrins, etc.), are increasingly being tested as PDT sensitisers.

By way of example, the reduction of porphyrins to form chlorins and bacteriochlorins leads to changes in optical properties which result in more efficient absorption in the red and near-infrared regions of the spectrum. There are a number of ways of synthesising chlorins and bacteriochlorins from porphyrins, including diimide reduction, cis-hydroxylation with osmium tetraoxide, and meso-β cyclisation.

To date, however, there are a number of disadvantages associated with known chlorins and bacteriochlorins. Firstly, they are synthetically challenging and their structural complexity often leads to a mixture of products. Secondly, they tend to exhibit poor water solubility which means that complex pharmaceutical formulations are required for systemic applications. Thirdly, their chemical instability reduces shelf life, and their negative or neutral overall charge often makes absorption by cells difficult.

In the context of PDT, fluorescence analysis and imaging, it is highly preferable to exert some degree of control over the localisation of the chromophore in vitro or in vivo. This is particularly important in PDT as the short lifetime of singlet oxygen means that in order to bring about the death of a target cell, the sensitiser must either be positioned immediately alongside or preferably within that cell.

Various attempts have been made to control the targeting of porphyrin sensitisers to particular target cells in vivo for the purpose of PDT. Recent efforts at achieving the specific attachment of porphyrin sensitisers to suitable delivery molecules have focused on covalent conjugation to proteins of biological importance, such as human and bovine serum albumins, monoclonal antibodies and lipoproteins. The majority of such bioconjugations have involved chlorin e6, mTHPC (Foscan) or sulphonated phthalocyanines as the sensitiser and have made use of carbodiimide or active ester based methodology. However, there are a number of drawbacks associated with the reactive multifunctional nature of these molecules which in turn can lead to cross-linking problems and non-covalent binding. A minimisation of these effects is desirable as they lead to a reduction in efficacy of the sensitiser.

The present invention seeks to provide new porphyrin derivatives which exhibit improved properties with regard to photodynamic therapy and/or medical imaging. Preferably, the present invention seeks to alleviate one or more of the above-mentioned problems associated with prior art PDT agents. The invention further seeks to provide valuable intermediates for making such porphyrin derivatives.

### SUMMARY OF THE INVENTION

A first aspect of the invention includes a compound of formula I, or salt thereof: 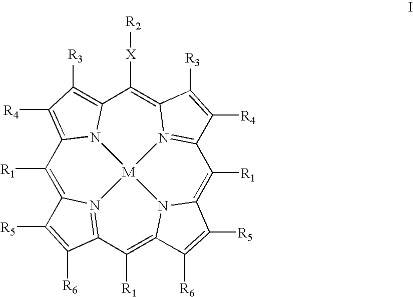
wherein
each R₁ is independently 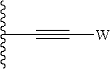
wherein W is an aryl, alkyl or heteroaryl group, at least some of which may be optionally substituted by one or more of:
OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, or a sugar derivative, Q;
   R₂ is H, a halogen, an isothiocyanate group, a haloacetamide, maleimide, Y-aryl or Y-heteroaryl, where Y is O, S, NH, C(O) or CO₂, and where said aryl or heteroaryl group may be optionally substituted by one or more of:
OH, halogen, an isothiocyanate group, a haloacetamide, maleimide, COOH, NO₂, NH₂, alkyl, haloalkyl, alkoxy, (CO)_{n′}(O)_{m′}Z′, a polyethylene glycol group, an alkyl sulfonate group, an alkyl-COOH group, a substituted or unsubstituted benzyl group, or a sugar derivative, Q′;
   Z and Z′ are each independently silicon-containing protecting groups and m, m′, n and n′ are each independently 0 or 1;
   X is a C₁₋₂₀ alkylene group (e.g., a C₁₋₁₀ alkylene group (e.g., a C₅₋₁₀ alkylene group)), optionally substituted by one or more substituents selected from halogen, NO₂, CN, OH, OMe, NH₂, CF₃, COOH and CONH₂;
   each R₃, R₄, R₅ and R₆ is independently H, alkyl, alkoxy, halogen or OH; and
   M is 2H or a metal.

In some embodiments, R₂ is H, halo or is selected from the following: 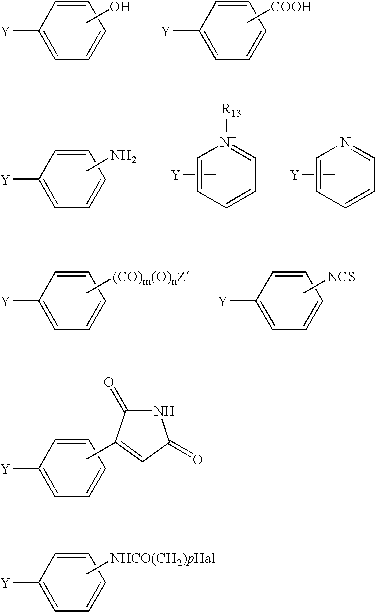
wherein R₁₃ is an alkyl group, an alkyl sulfonate group, an alkyl-COOH group or a substituted or unsubstituted benzyl group, and p is an integer from 1 to 10. In some embodiments, R₂ is H, a halogen, or a Y-aryl.

In some embodiments, R₂ is selected from the following:

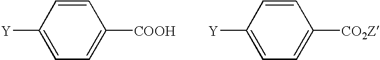

In some embodiments, silicon-containing protecting group, Z′, is CH₂)_{q′}Si(R₇)(R₈)(R₉), where R₇, R₈ and R₉ are each independently hydrocarbyl groups and q′ is 0, 1, 2, 3, 4 or 5. One or all of R₇, R₈ and R₉ can, independently, be alkyl groups. Z′ can be CH₂CH₂SiMe₃.

In some embodiments R₂ is selected from the following:

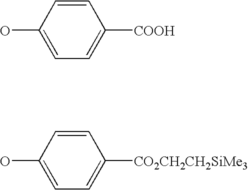

In some embodiments, each R₁ is independently 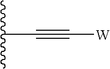
where W is an aryl or heteroaryl group, at least some of which can be optionally substituted by one or more of:
OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, and a sugar derivative.

In some embodiments, W is an optionally substituted phenyl group.

In some embodiments, W is an optionally substituted pyridyl group.

In some embodiments, W is selected from the following: 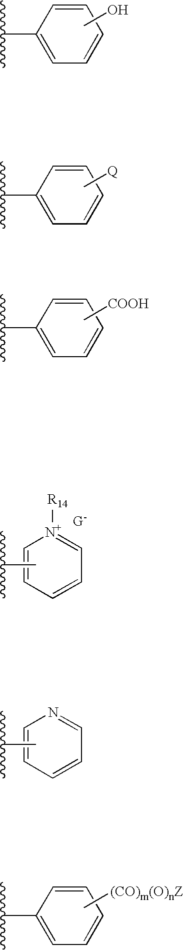
where R¹⁴ is an alkyl group, an alkyl sulfonate group, an alkyl-COOH group or a substituted or unsubstituted benzyl group, and G⁻ is a counter ion. In some embodiments, Q is D-mannopyranoside or a derivative thereof.

In some embodiments, W is selected from the following:

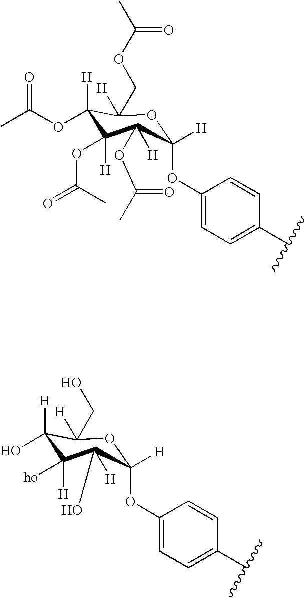

In some embodiments, silicon-containing protecting group, Z, is (CH₂)_{q}Si(R₁₀)(R₁₁)(R₁₂), wherein R₁₀, R₁₁ and R₁₂ are each independently hydrocarbyl groups, and q is 0, 1, 2, 3, 4 or 5. At least one (e.g., all) of R₁₀, R₁₁ and R₁₂ can, independently, be alkyl groups.

In some embodiments, W is selected from the following: 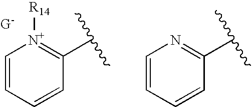
and G⁻ is halide or p-toluene sulfonate.

In some embodiments, M is selected from 2H, Ni, Pb, V, Pd, Co, Nb, Al, Sn, Zn, Cu, Mg, Ca, In, Ga, Fe, Eu, Lu, Pt, Ru, Mn and Ge. In some embodiments, M is 2H or Zn.

In some embodiments, the compound is:

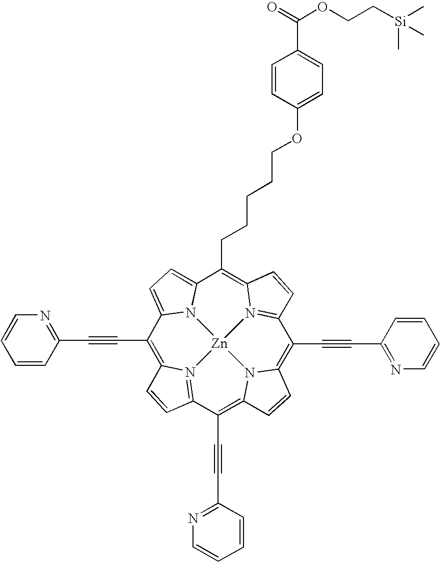

In some embodiments, the compound is:

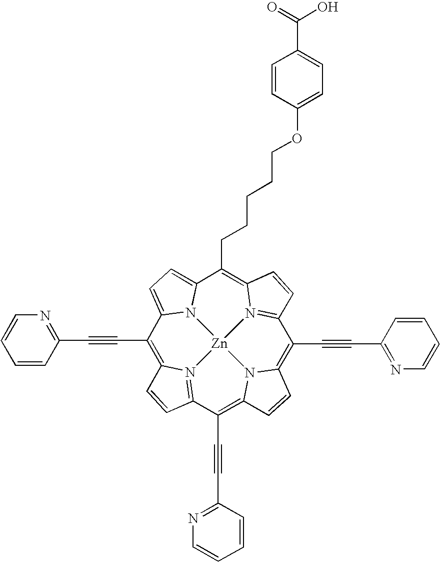

In some embodiments, the compound is:

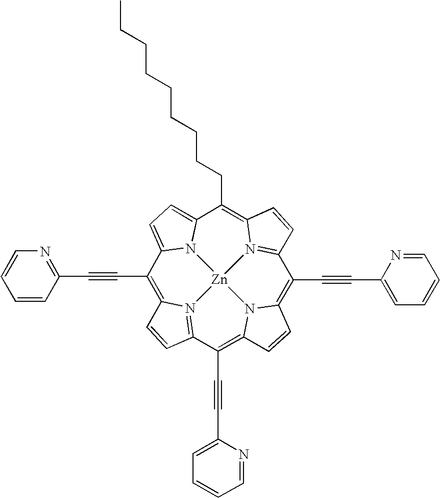

In some embodiments, the compound is:

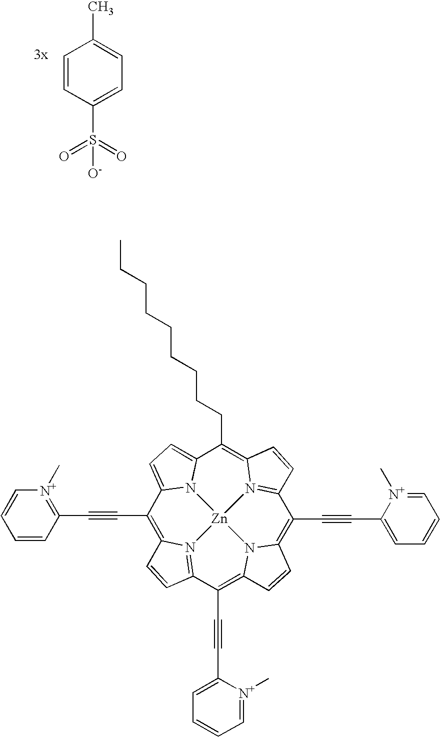

In some embodiments, the compound is:

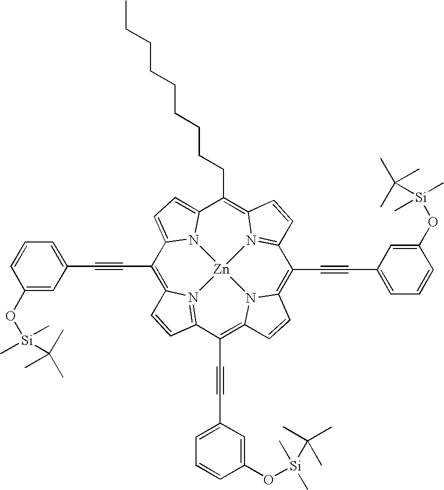

In some embodiments, the compound is:

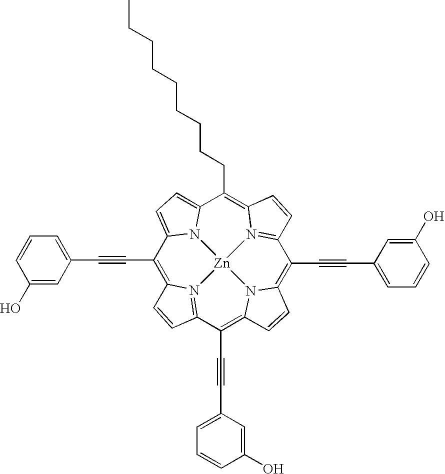

In some embodiments, the compound is:

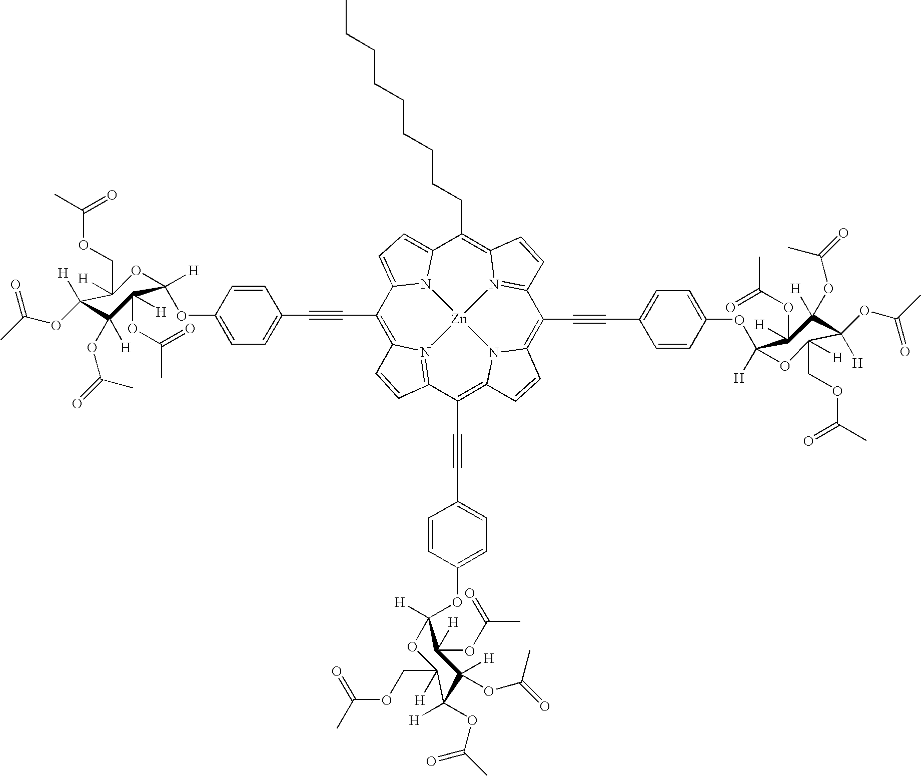

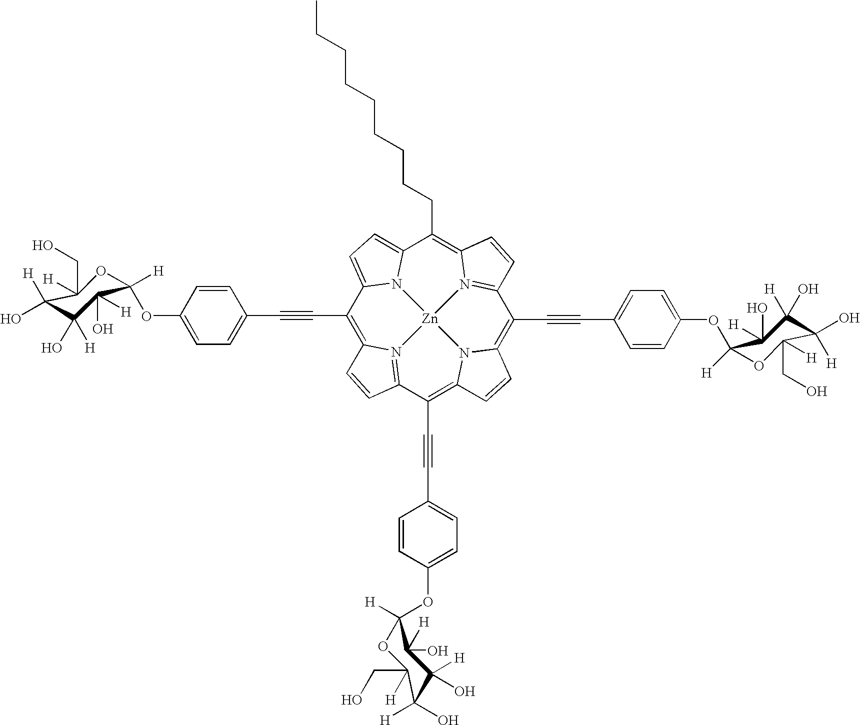

In some embodiments, the compound is:

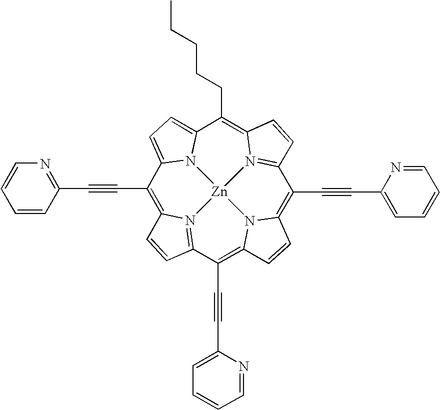

In some embodiments, the compound is:

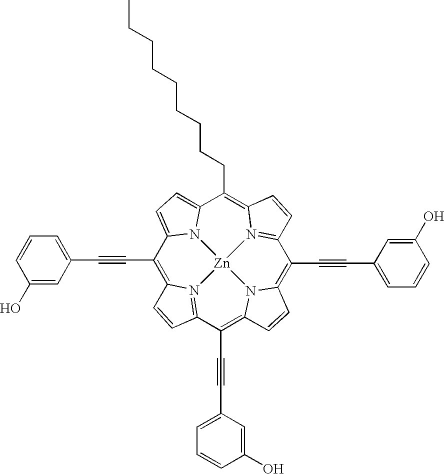

Another aspect of the invention relates to a compound of formula Ia, or salt thereof: 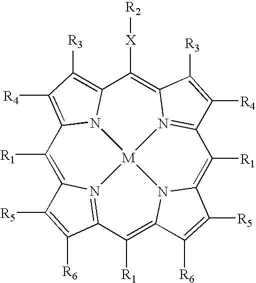
Ia
Where:
each R₁ is independently H or halogen;
R₂ is a halogen, an isothiocyanate group, a haloacetamide, maleimide, Y-aryl or Y-heteroaryl, where Y is O, S, NH, C(O) or CO₂, and where said aryl or heteroaryl group may be optionally substituted by one or more of:
OH, halogen, an isothiocyanate group, a haloacetamide, maleimide, COOH, NO₂, NH₂, alkyl, haloalkyl, alkoxy, (CO)_{n′}(O)_{m′}Z′, a polyethylene glycol group, an alkyl sulfonate group, an alkyl-COOH group, a substituted or unsubstituted benzyl group, or a sugar derivative;
   Z′ is a silicon-containing protecting group and m′ and n′ are each independently 0 or 1;
   X is a C₁₋₂₀ alkylene group, optionally substituted by one or more substituents selected from halogen, NO₂, CN, OH, OMe, NH₂, CF₃, COOH and CONH₂;
   each R₃, R₄, R₅ and R₆ is independently H, alkyl, alkoxy, halogen or OH; and
   M is 2H or a metal;
   with the proviso that:
(a) when R₃ and R₅ are Me, R₄ and R₆ are Et, and R₁ is H, X—R₂ is other than CH₂OPh, CH₂CH₂NO₂ or CH₂CH(OMe)₂; and
(b) when R₃₋₆ are Et, and R₁ is H, X—R₂ is other than CH(CF₃)OH.

In some embodiments, each R₁ is either H or iodo.

In some embodiments, X, R₂ and/or M are as defined for compound I.

In some embodiments, the compound is selected from the following:

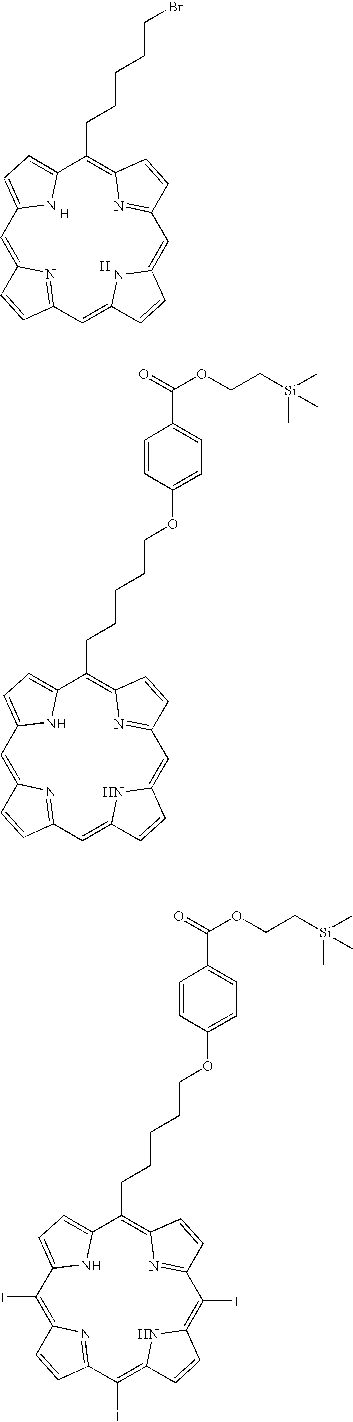

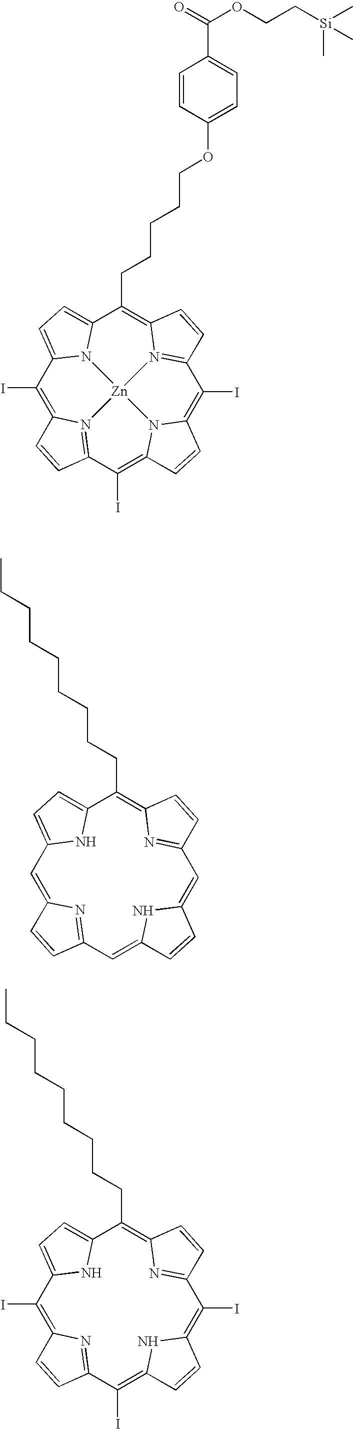

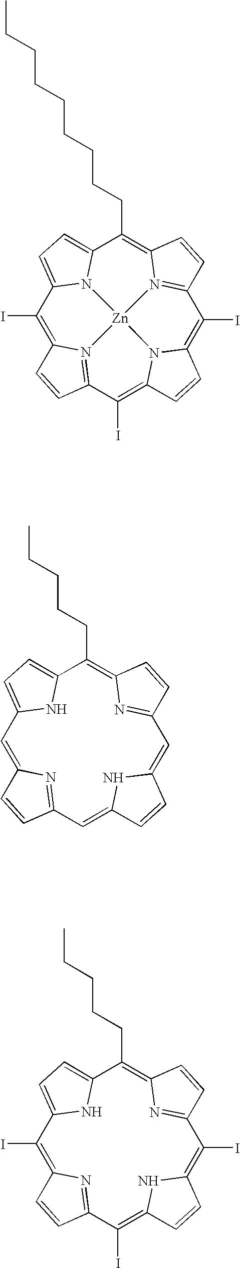

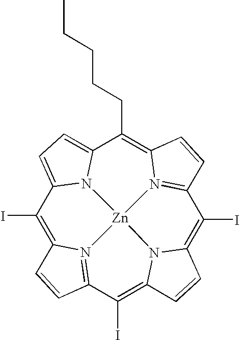

Another aspect of the invention relates to a method for preparing one or more of the compounds described herein.

Another aspect of the invention relates to a composition including one or more of the compounds described herein optionally admixed with a pharmaceutically acceptable diluent, excipient and/or carrier.

Another aspect of the invention relates to a composition including one or more of the compounds described herein and a targeting moiety selected from a recombinant antibody, a Fab fragment, a F(ab′)₂ fragment, a single chain Fv, a diabody, a disulfide linked Fv, a single antibody domain and a CDR.

Another aspect of the invention relates to a composition including a conjugate molecule including a polypeptide carrier which includes at least one alpha helix having synthetically attached thereto a plurality of porphyrins of as any of the compounds herein.

Another aspect of the invention relates to a method for preparing a composition including a conjugate molecule.

Another aspect of the invention relates to a method for using a composition including a conjugate in a medical setting (e.g., in a medical imaging procedure).

Another aspect of the invention relates to a method of using one or more compounds herein (including conjugates) in a photodynamic therapy and/or to prepare a medicament for photodynamic therapy.

Another aspect of the invention relates to a method of using one or more compounds herein (including conjugates) to treat a proliferative disorder (e.g., cancer) by administration of the one or more compounds.

Another aspect of the invention relates to a method for preparing a compound of formula I: 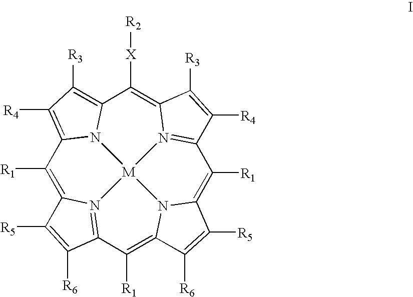
by a method including reacting a compound of formula II with a dipyrrole of formula III to form a compound of formula Ib, in which R₁ is H, and X, R₂ are as defined above,

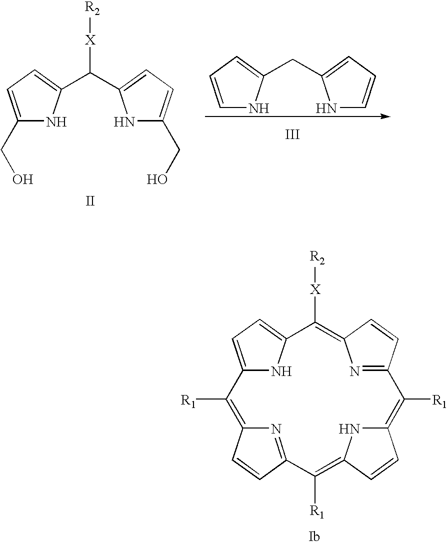

The method may further include the step of converting the compound of formula Ib to a compound of formula Ic, in which R₁ is halogen, X, R₂ are as defined above.

The method may further include reacting the compound of formula Ic with 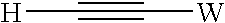
to form a compound of formula Id wherein R₁ is 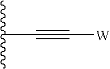
and X, R₂ and W are defined as above.

### BRIEF DESCRIPTION OF THE DRAWINGS

FIG. 1 illustrates a targetable-carrier protein.

FIGS. 2A and 2B illustrate structure of carrier proteins.

FIGS. 2C and 2D illustrate a 4-helix bundle, which can be used to deliver porphyrins.

FIG. 3 illustrates the construction of an scFv-4-helix bundle fusion gene.

FIG. 4 illustrates over-expression anti-CEA scFv (lanes 5-7) and scFv4 helix bundle (lanes 1-4) fusion protein in E. coli BL21(DE3).

FIG. 5 illustrates the absorption spectrum of porphyrin.

FIG. 6A illustrates a plot of mV against power (mV) for compound [11].

FIG. 6B illustrates the UV-visible spectrum (absorbance versus wavelength) for compound [11].

FIG. 6C illustrates a plot of mV against power (mV) for reference 1 (chlorophyll A).

FIG. 6D illustrates the UV-visible spectrum (absorbance versus wavelength) for chlorophyll A.

### DETAILED DESCRIPTION

As mentioned above, in a first aspect the invention provides compounds of formula I.

In a preferred aspect, the present invention relates to a compound of formula Ie 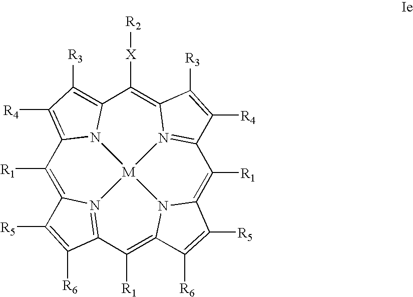
wherein
each R₁ is independently 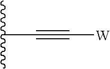
wherein W is an aryl, alkyl or heteroaryl group, each of which may be optionally substituted by one or more of:
OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, or a sugar derivative;
   R₂ is a halogen, an isothiocyanate group, a haloacetamide, maleimide, Y-aryl or Y-heteroaryl, where Y is O, S, NH, C(O) or CO₂, and where said aryl or heteroaryl group may be optionally substituted by one or more of:
OH, halogen, an isothiocyanate group, a haloacetamide, maleimide, COOH, NO₂, NH₂, alkyl, haloalkyl, alkoxy, (CO)_{n′}(O)_{m′}Z′, a polyethylene glycol group, an alkyl sulfonate group, an alkyl-COOH group, a substituted or unsubstituted benzyl group, or a sugar derivative;
   Z and Z′ are each independently silicon-containing protecting groups and m, m′, n and n′ are each independently 0 or 1;
   X is a C₁₋₂₀ alkylene group, optionally substituted by one or more substituents selected from halogen, NO₂, CN, OH, OMe, NH₂, CF₃, COOH and CONH₂;
   R₃, R₄, R₅ and R₆ are each independently H, alkyl, alkoxy, halogen or OH; and
   M is 2H or a metal.

As used herein, the term “hydrocarbyl” refers to a group comprising at least C and H that may optionally comprise one or more other suitable substituents. Examples of such substituents may include halo-, alkoxy-, nitro-, an alkyl group, or a cyclic group. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon.

As used herein, the term “alkyl” refers to a saturated carbon-containing chain which may be straight or branched, and substituted (mono- or poly-) or unsubstituted. Preferably, the alkyl group is a branched or unbranched C₁₋₃₀ alkyl group, more preferably an unbranched C₁₋₂₀ alkyl group, even more preferably a C₁₋₁₀ or C₁₋₅ alkyl group. Suitable substituents may include, for example, halo, NO₂, NH₂, CF₃, alkoxy, OH, CONH₂ and COOH.

Accordingly, the term “haloalkyl” refers to an alkyl group substituted by a halogen, for example, chlorine, bromine, fluorine or iodine.

As used herein, the term “aryl” refers to a substituted (mono- or poly-) or unsubstituted monoaromatic or polyaromatic system, wherein said polyaromatic system may be fused or unfused.

As used herein, the term “heteroaryl” refers to an aromatic heterocycle comprising one or more heteroatoms and which may be substituted (mono- or poly-) or unsubstituted. Said heteroaryl group may be a monoaromatic or polyaromatic system, wherein said polyaromatic system may be fused or unfused. Preferred heteroaryl groups include pyrrole, pyrimidine, pyrazine, pyridine, quinoline and furan.

As used herein, the term “alkylene” refers to both linear and cyclic alkylene groups, each of which may be substituted or unsubstituted.

Suitable substituents for said alkylene, heteroaryl, benzyl and aryl groups include, for example, alkyl, halo, NO₂, NH₂, CN, CF₃, alkoxy, OH, CONH₂, and COOH.

As used herein, the term “sugar derivative” encompasses mono-, di- and tri-saccharides, and derivatives, epimers and enantiomers thereof.

Monosaccharides have the general formula (CH₂O)ₙ and can exist as either straight chain or ring-shaped molecules. They are classified according to the number of carbon atoms they possess; trioses have three carbons (n=3), tetroses four (n=3), pentoses five (n=3) and hexoses six (n=3). Each of these subgroups may be further divided into aldoses and ketoses, depending on whether the molecule contains an aldehyde group (—CHO) or a ketone group (C═O). Suitable derivatives include monosaccharides in which one or more of the —OH groups are acylated, i.e. where one or more of the —OH groups are in the form of —OCOR groups, preferably where R is Me.

Preferably, the sugar derivative is linked to the aryl, alkyl or heteroaryl group via one of the oxygen atoms of the sugar derivative. More preferably, the sugar derivative is linked to the aryl, alkyl or heteroaryl group via a direct —O— bond to one of the oxygen atoms of the sugar derivative.

Monosaccharides can exist in open chain form or as intramolecular hemiacetals (pyranoses) or intramolecular hemiketals (furanoses). By way of example, the C-1 aldehyde group of the open chain form of glucose can react with the C-5 hydroxyl group to form α-D-glucopyranose or β-D-glucopyranose. Similarly, the C-2 keto group in the open chain form of fructose can react with the C-5 hydroxyl group to form α-D-fructofuranose or β-D-fructofuranose.

Suitable monosaccharides include, for example, D- or L-glyceraldehyde, dihydroxyacetone, D-erythrose, D-threose, D-ribose, D-arabinose, D-xylose, D-lyxose, D-glucose, D-fructose, D-galactose, D-allose, D-altrose, D-mannose, D-gulose, D-idose, D-talose, D-erythrulose, D-ribulose, D-xylulose, D-psicose, D-sorbose, D-tagatose, α-D-glucopyranose, β-D-glucopyranose, α-D-mannopyranose, α-D-fructopyranose, β-D-fructopyranose, α-D-fructofuranose, β-D-fructofuranose, β-D-ribofuranose, and β-D-2-deoxyribofuranose.

Disaccharides consist of two linked monosaccharide molecules, and include, for example, maltose, sucrose and lactose. Likewise, trisaccharides consist of three linked monosaccharide molecules.

In one particularly preferred embodiment of the invention, sugar derivatives Q and Q′ are each independently α-D-mannopyranose or penta-O-acetyl-α-D-mannopyranose.

As used herein, the symbol [symbol] denotes the point of attachment to the molecule.

In one preferred embodiment of the invention, X is a C₁₋₁₀ alkylene group.

In one preferred embodiment of the invention, X is a C₅₋₁₀ alkylene group. More preferably still, X is a C₅ or C₉ alkylene group.

In another preferred embodiment, X is a C₁₋₅ alkylene group.

In the case where R₂ is Y-aryl or Y-heteroaryl substituted by a polyethylene glycol group (PEG), or W is substituted by a polyethylene glycol group, the polyether typically has a molecular weight of 2000 to 5000 daltons. The polyether may be etherified or esterified at the terminal hydroxy group, and is more preferably etherified or esterified with a methyl group.

More preferably still, R₂ is H, halo or is selected from the following: 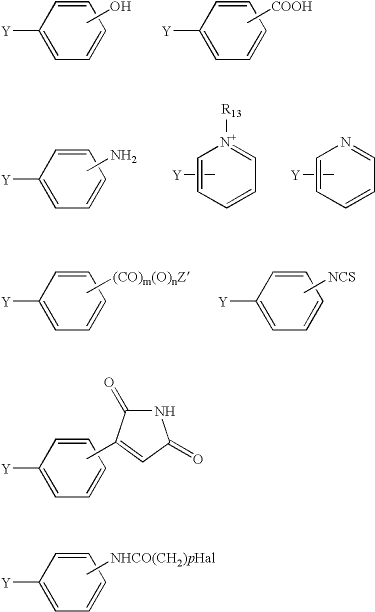
wherein R₁₃ is an alkyl group, an alkyl sulfonate group, an alkyl-COOH group or a substituted or unsubstituted benzyl group, and p is an integer from 1 to 10. Suitable benzyl group substituents include, for example, alkyl, halo, NO₂, NH₂, CN, CF₃, alkoxy, OH, CONH₂, and COOH.

Preferably, R₁₃ is a C₁₋₆ alkyl sulfonate group, more preferably a propylsulfonate or a butylsulfonate group.

In one particularly preferred embodiment, R₂ is selected from the following: 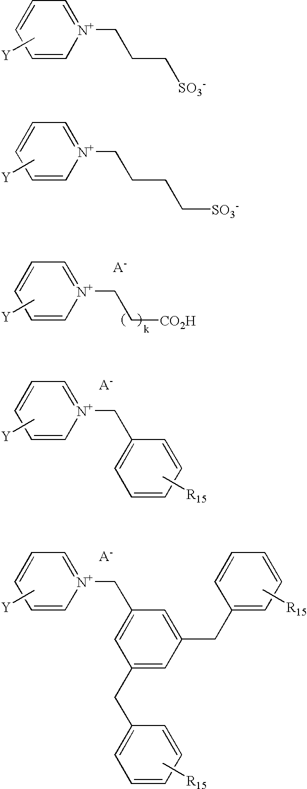
where A⁻ is a counter ion, for example, a halide counter ion such as iodide, or more preferably chloride, k is an ineteger from 1 to 10, and R₁₅ is a substituent selected from alkyl, halogen, NO₂, CN, OH, OMe, NH₂, CF₃, COOH and CONH₂.

Compounds in which R₁₃ is an alkyl-COOH group may be obtained by reacting the pyridinyl compound with the corresponding bromo- or iodo-alkyl acid.

Compounds in which R₁₃ is a substituted or unsubstituted benzyl group may be obtained by reacting the pyridinyl compound with a benzyl bromide or a substituted benzyl bromide.

In another preferred embodiment of the invention, R₂ is H, a halogen, or Y-aryl.

In another particularly preferred embodiment, R₂ is selected from the following:

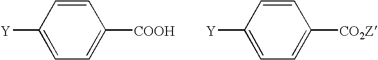

In one particularly preferred embodiment, Y is O.

Suitable silicon-containing protecting groups will be familiar to the skilled artisan (see for example, “Protective Groups in Organic Synthesis” by Peter G. M. Wuts and Theodoro W. Greene, 2^{nd} Edition).

In one preferred embodiment, R₂ is a Y-aryl group bearing a protected OH group OZ′, wherein Z′ is a trialkyl silyl group. Preferably, said trialkyl silyl group is a trimethylsilyl group (SiMe₂), a triethylsilyl group (SiEt₃), a teritiary-butyldimethylsilyl (TBDMS) group (Si(Me)₂CMe₃), an iso-propyldimethylsilyl group (Si(Me)₂CHMe₂), a phenyldimethylsilyl group (Si(Me)₂Ph), a di-tertiary-butylmethylsilyl (DTBMS) group (^{t}Bu₂MeSi) or a tri-isopropylsilyl (TIPS) group (SiⁱPr₃).

In one preferred embodiment, R₂ is a Y-aryl group bearing a COOZ′ group. Preferably, said silicon-containing protecting group, Z′, is (CH₂)_{q′}Si(R₇)(R₈)(R₉), wherein R⁷, R⁸ and R⁹ are each independently hydrocarbyl groups and q′ is 0, 1, 2, 3, 4 or 5.

Preferably, R₇, R₈ and R₉ are each independently alkyl groups.

Preferably, COOZ′ is a silyl ester in which there is a direct silicon-oxygen bond (i.e. where q is 0). For this embodiment, typically COOZ′ is a trimethylsilyl ester group (COOSiMe₂), a triethylsilyl ester group (COOSiEt₃), a teritiary-butyldimethylsilyl (TBDMS) ester group (COOSi(Me)₂CMe₃), a iso-propyldimethylsilyl ester group (COOSi(Me)₂CHMe₂), a phenyldimethylsilyl ester group (COOSi(Me)₂Ph), a di-tertiary-butylmethylsilyl (DTBMS) ester group (COOSi^{t}Bu₂Me) or a tri-isopropylsilyl (TIPS) ester group (COOSiⁱPr₃).

In another preferred embodiment, COOZ′ is a silyl ester in which there is a silicon-carbon bond (i.e. q is other than zero). By way of example, for this embodiment, Z′ may be a 2-(trimethylsilyl)-ethoxymethyl (SEM) ester group (COOCH₂OCH₂CH₂SiMe₃), a tri-iso-propylsilylmethyl ester group (COOCH₂SiⁱPr₃), or a 2-(trimethylsilyl)ethyl (TMSE) ester group (COOCH₂CH₂SiMe₃).

In one particularly preferred embodiment, COOZ′ is COOCH₂CH₂SiMe₃.

In one particularly preferred embodiment, R₂ is selected from:

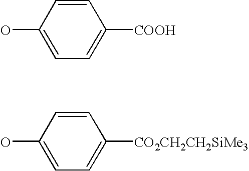

Preferably, R₃, R₄, R₅ and R₆ are all H.

In one preferred embodiment, each R₁ is independently 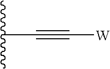
wherein W is an aryl or heteroaryl group, each of which may be optionally substituted by one or more of:
OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, and a sugar derivative.

Meso-arylethynyl substituted porphyrins are particularly advantageous in PDT as the whole UV/visible spectrum is red-shifted by as much as 50 nm. The intensity of the Q bands is also increased relative to the B band. This turns the characteristic porphyrin red colour to a brilliant green: hence the trivial name, chlorphyrin-green porphyrin. Chlorphyrins have better red-light absorbing properties than porphyrins, and are expected to be well-suited for PDT. Furthermore, chlorphyrins show excellent triplet-state yields and lifetimes.

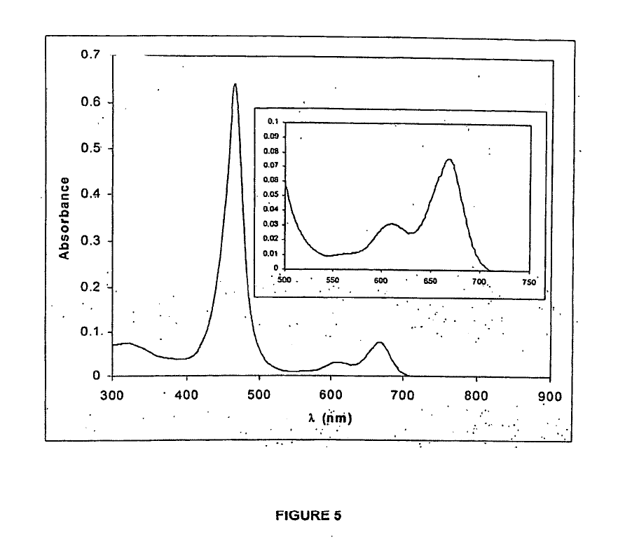

Thus, the compounds of this preferred embodiment exhibit strong absorption at longer wavelengths, for example, between 650 and 800 nm (see FIG. 5). This shift in absorption further into the red is attributable to the extended pi system arising from the conjugation of the alkynyl groups to the porphyrin nucleus. The compounds of this embodiment allow for deeper tissue penetration, whilst at the same time exhibiting a surprisingly high quantum yield for singlet oxygen. By way of example, zinc-5-[5-(2′-(trimethylsilyl)ethyl-4-hydroxybenzoate)pentane]-10,15,20-tri-2-ethynylpyridine-porphyrin, compound [11], exhibits a singlet oxygen quantum yield of around 0.81 compared to compounds known in the art, such as disulfonated aluminium phthalocyanine (AIPCS2) and meta-(tetrahydroxyphenyl)-chlorin (mTHPC) which exhibit singlet oxygen quantum yields of 0.3 and 0.43 respectively.

In one particularly preferred embodiment, W is an optionally substituted phenyl group. In another particularly preferred embodiment, W is an optionally substituted pyridyl group.

More preferably, W is selected from the following: 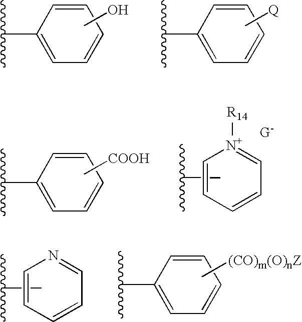
wherein R¹⁴ is an alkyl group, an alkyl sulfonate group, an alkyl-COOH group or a substituted or unsubstituted benzyl group, and Z may be the same or different to Z′ as defined above, and G⁻ is a counter ion. Suitable benzyl group substituents include, for example, alkyl, halo, NO₂, NH₂, CN, CF₃, alkoxy, OH, CONH₂, and COOH.

Preferably, R₁₄ is defined as above for R₁₃ and may be the same or different to R₁₄.

In one preferred embodiment, Q is D-mannopyranoside or a derivative thereof.

In one particularly preferred embodiment, W is selected from the following:

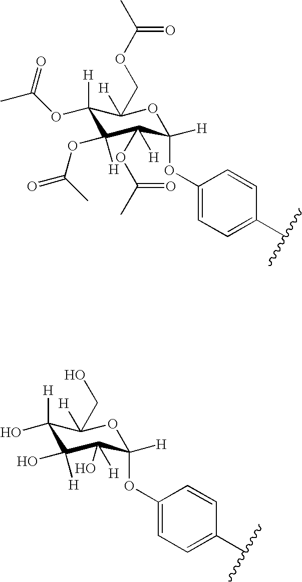

In another particularly preferred embodiment, W is selected from the following: 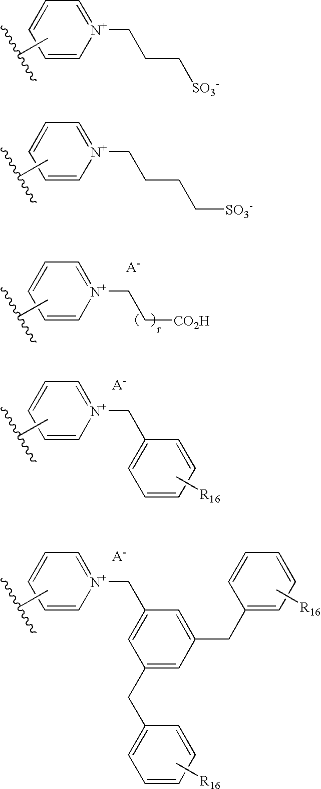
where A⁻ is a counter ion, for example, a halide counter ion such as iodide, or more preferably chloride, r is an ineteger from 1 to 10, and R₁₆ is a substituent selected from alkyl, halogen, NO₂, CN, OH, OMe, NH₂, CF₃, COOH and CONH₂.

Compounds in which R₁₄ is an alkyl-COOH group may be obtained by reacting the pyridinyl compound with the corresponding bromo- or iodo-alkyl acid.

Compounds in which R₁₆ is a substituted or unsubstituted benzyl group may be obtained by reacting the pyridinyl compound with the corresponding benzyl bromide.

In a preferred embodiment, said silicon-containing protecting group, Z, is (CH₂)_{q}Si(R₁₀)(R₁₁)(R₁₂), wherein R₁₀, R₁₁ and R₁₂ are each independently hydrocarbyl groups and q is 0, 1, 2, 3, 4 or 5.

More preferably, R₁₀, R₁₁ and R₁₂ are each independently alkyl groups.

In a particularly preferred embodiment, W is an aryl group bearing a COOZ substituent, wherein COOZ is COOCH₂CH₂SiMe₃.

In one especially preferred embodiment, W is selected from: 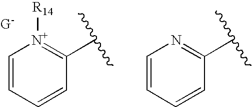
and G⁻ is halide or p-toluene sulfonate.

The compounds of formula I can be metallated or in their free base form. Preferably, M is selected from 2H, Ni, Pb, V, Pd, Co, Nb, Al, Sn, Zn, Cu, Mg, Ca, In, Ga, Fe, Eu, Lu, Pt, Ru, Mn and Ge. More preferably, M is 2H or Zn.

Particularly preferred compounds of the invention are as follows: 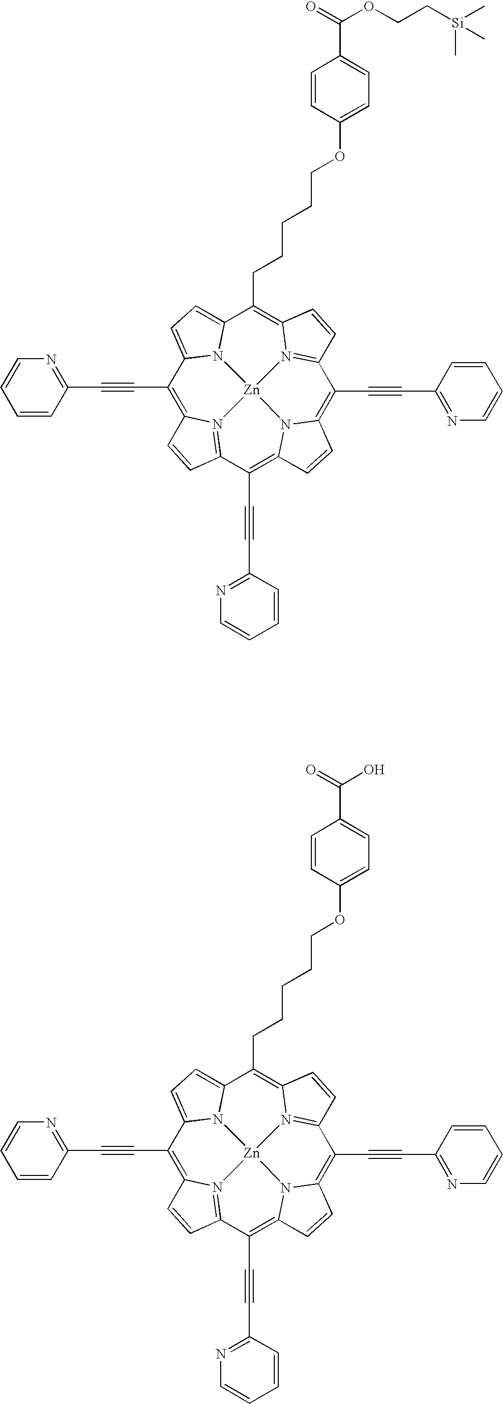 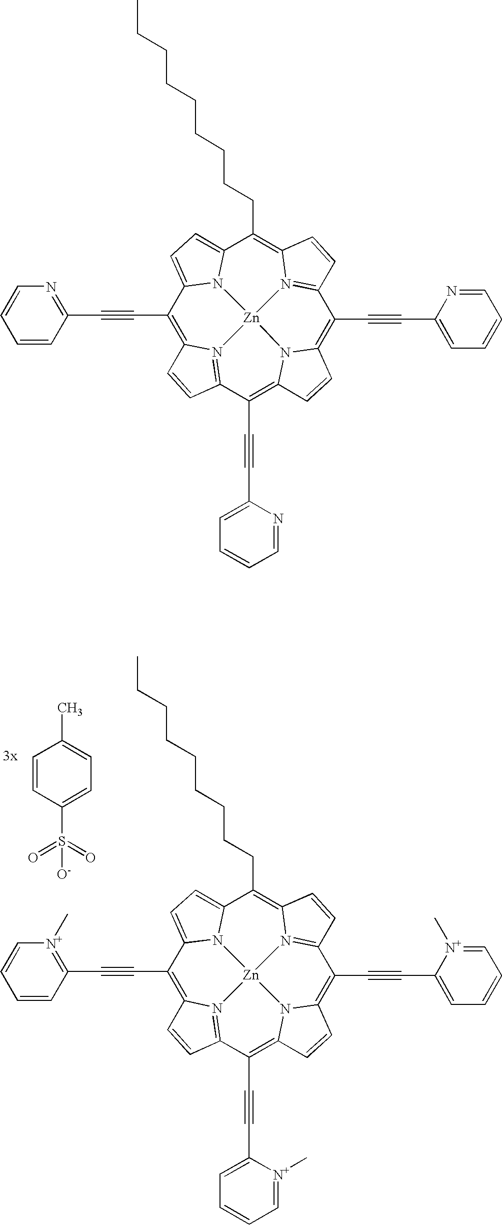 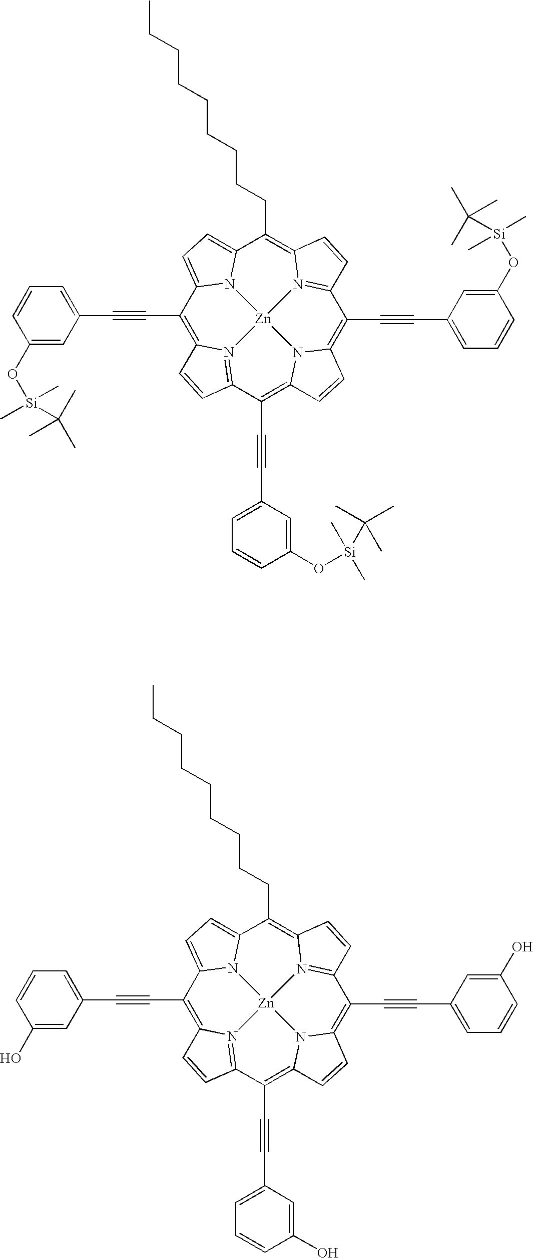 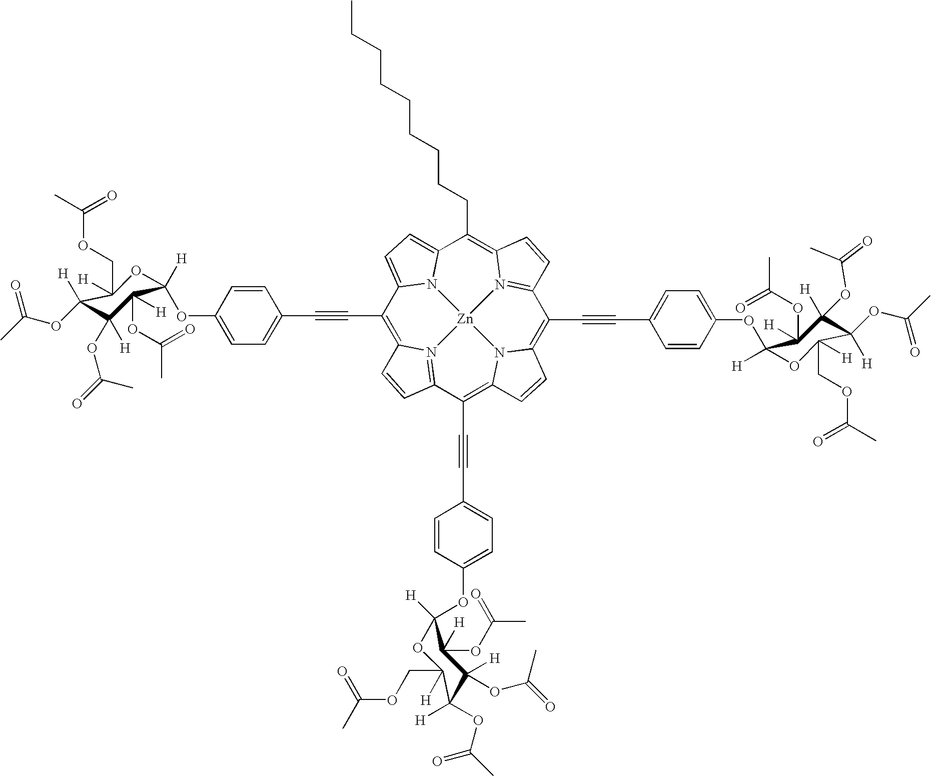 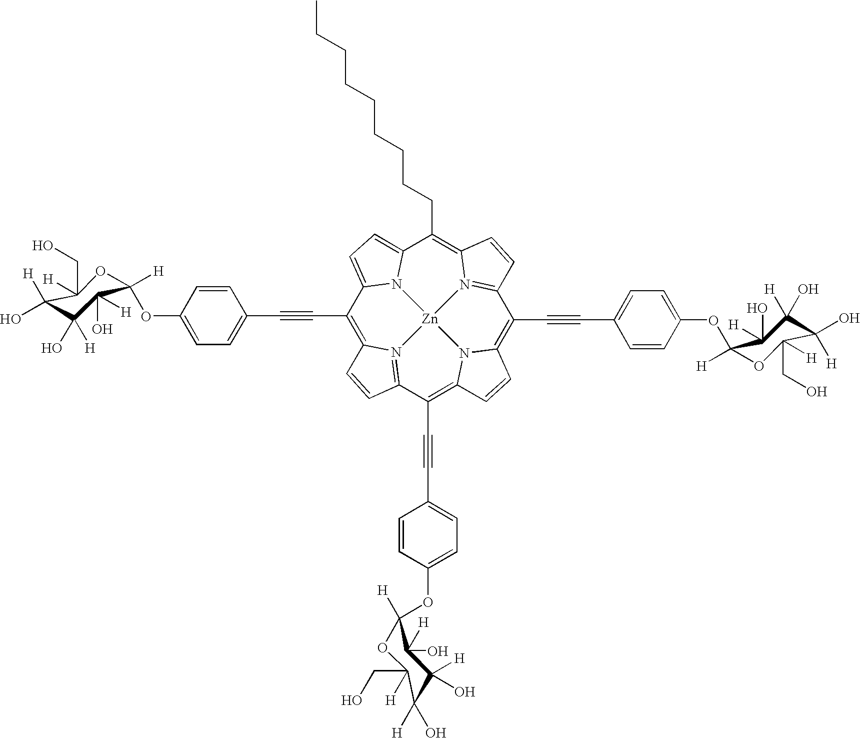 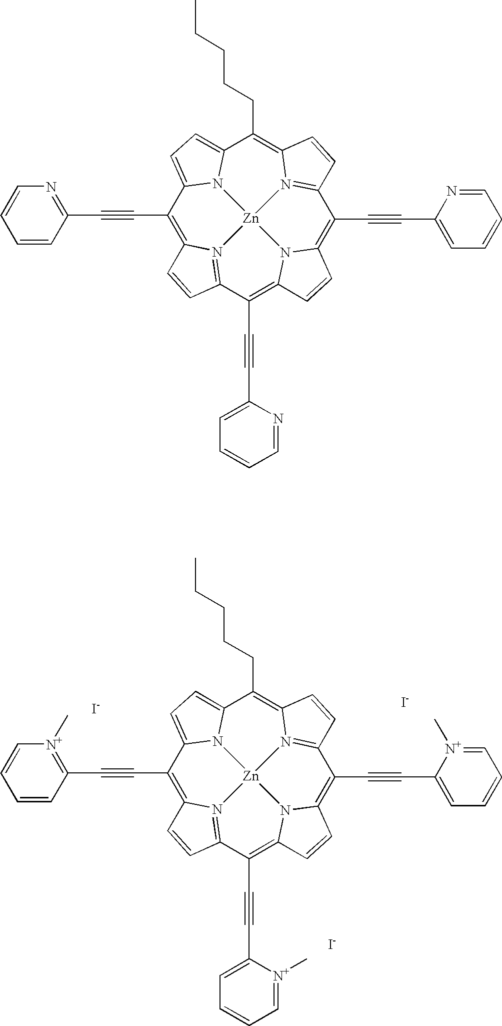
Intermediates

The present invention further relates to a series of intermediate compounds useful in the preparation of compounds of formula I as defined above.

Thus, another aspect of the invention relates to a compound of formula Ia 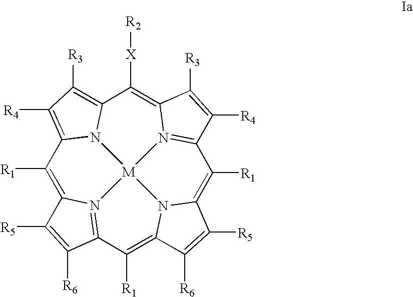
wherein
each R₁ is independently H or halo;
R₂ is a halogen, an isothiocyanate group, a haloacetamide, maleimide, Y-aryl or Y-heteroaryl, where Y is O, S, NH, C(O) or CO₂, and where said aryl or heteroaryl group may be optionally substituted by one or more of:
OH, halogen, an isothiocyanate group, a haloacetamide, maleimide, COOH, NO₂, NH₂, alkyl, haloalkyl, alkoxy, (CO)ₙ′(O)_{m′}Z, a polyethylene glycol group, an alkyl sulfonate group, an alkyl-COOH group, a substituted or unsubstituted benzyl group, or a sugar derivative′;
   Z′ is a silicon-containing protecting group and m′ and n′ are each independently 0 or 1;
   X is a C₁₋₂₀ alkylene group, optionally substituted by one or more substituents selected from halogen, NO₂, CN, OH, OMe, NH₂, CF₃, COOH and CONH₂;
   each R₃, R₄, R₅ and R₆ is independently H, alkyl, alkoxy, halogen or OH; and
   M is 2H or a metal;
   with the proviso that:
(a) when R³ and R⁵ are Me, R⁴ and R⁶ are Et, and R¹ is H, X—R² is other than CH₂OPh, CH₂CH₂NO₂ or CH₂CH(OMe)₂; and
(b) when R³⁻⁶ are Et, and R¹ is H, X—R² is other than CH(CF₃)OH.

In one preferred embodiment, for compounds of formula Ia, X is a C₁₋₂₀ alkylene group, optionally substituted by one or more substituents selected from halogen, CN, NH₂, COOH and CONH₂.

In one particularly preferred embodiment of the invention, X is a C₅₋₁₀ alkylene group.

In a preferred embodiment, R₁ is H or iodo.

Preferably, X, R₂ and M are as defined above for compounds of formula I.

In one particularly preferred embodiment, said compound of formula Ia is selected from the following:

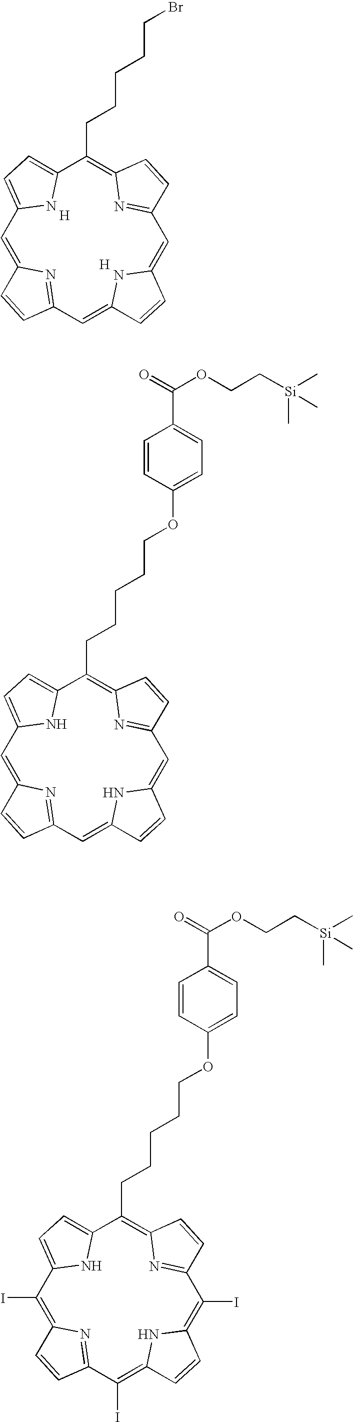

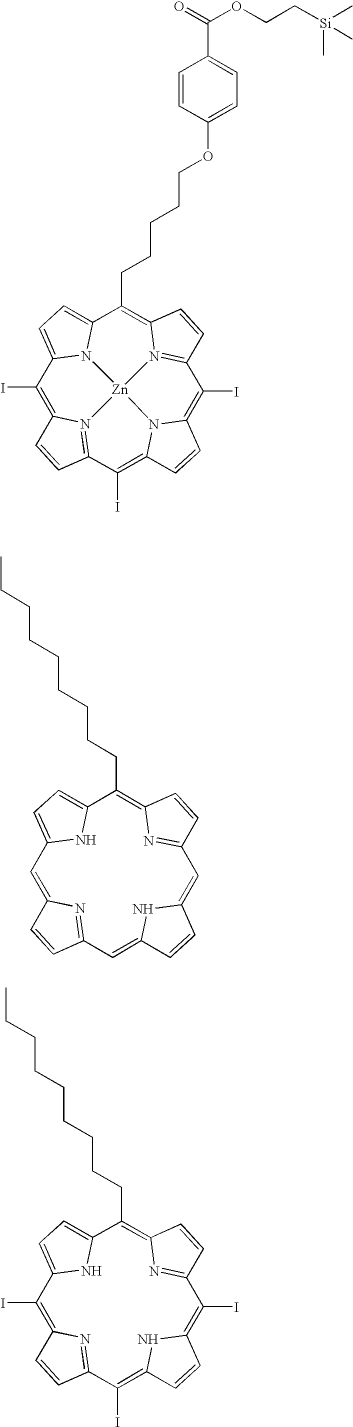

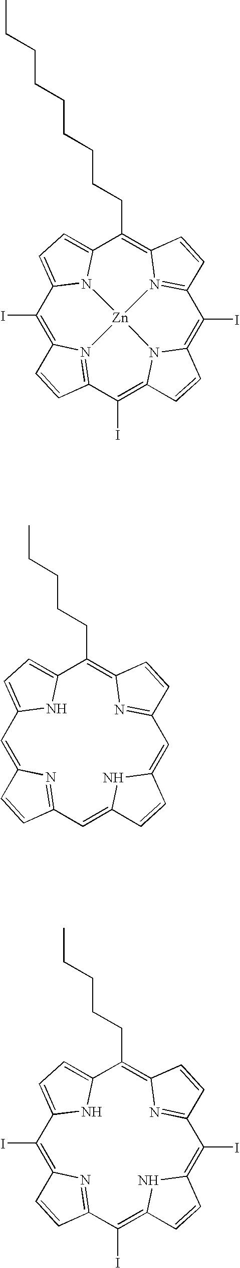

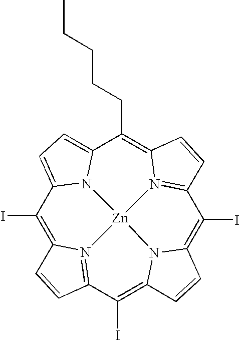

Another aspect of the invention relates to the use of compounds of formula Ia in the preparation of compounds of formula I as defined above. Further details regarding the synthetic preparation of the compounds of the invention may be found below under the heading “Synthesis”.

Conjugates

In another aspect, the invention relates to a conjugate molecule comprising a compound according to the invention and a targeting element.

As mentioned above, previous efforts at achieving the specific attachment of porphyrin sensitisers to suitable delivery molecules have focused on covalent conjugation to proteins. However, the reactive multifunctional nature of these molecules often leads to cross-linking problems and non-covalent binding. In a preferred embodiment, the present invention seeks to alleviate the aforementioned problems by providing porphyrin molecules bearing a single functional group capable of reacting with an amine or thiol residue on the protein. In this way, it is possible to exert better control over the extent of cross-linking. By way of example, using a meso-monoalkyl substituted porphyrin bearing a single carboxyl group (or other reactive functional group), it is possible to couple the porphyrin to a protein in a more controlled manner than with the corresponding di-, tri- or tetra-carboxy substituted porphyrin derivatives which often result in the formation of undesirable polymeric mixtures.

In a preferred embodiment, the compound of formula I comprises a haloacetamide group which is capable of cross-linking to the thiol group of a cysteine residue in a protein.

In another preferred embodiment, the compound of formula I comprises a maleimide group which is capable of cross-linking to the thiol group of a cysteine residue in a protein.

Preferably, the targeting element is selected from a recombinant antibody, a Fab fragment, a F(ab′)₂ fragment, a single chain Fv, a diabody, a disulfide linked Fv, a single antibody domain and a CDR.

As used herein, the term “CDR” or “complementary determining region” refers to the hypervariable regions of an antibody molecule, consisting of three loops from the heavy chain and three from the light chain, that together form the antigen-binding site. By way of example, the antibody may be selected from Herceptin, Rituxan, Theragyn (Pemtumomab), Infliximab, Zenapex, Panorex, Vitaxin, Protovir, EGFR1 or MFE-23.

In one preferred embodiment, the targeting element is a genetically engineered fragment selected from a Fab fragment, a F(ab′)₂ fragment, a single chain Fv, or any other antibody-derived format.

Conventionally, the term “Fab fragment” refers to a protein fragment obtained (together with Fc and Fc′ fragments) by papain hydrolysis of an immunoglobulin molecule. It consists of one intact light chain linked by a disulfide bond to the N-terminal part of the contiguous heavy chain (the Fd fragment). Two Fab fragments are obtained from each immunoglobulin molecule, each fragment containing one binding site. In the context of the present invention, the Fab fragment may be prepared by gene expression of the relevant DNA sequences.

Conventionally, the term “F(ab′)₂” fragment refers to a protein fragment obtained (together with the pFc′ fragment) by pepsin hydrolysis of an immunoglobulin molecule. It consists of that part of the immunoglobulin molecule N-terminal to the site of pepsin attack and contains both Fab fragments held together by disulfide bonds in a short section of the Fc fragment (the hinge region). One F(ab′)₂ fragment is obtained from each immunoglobulin molecule; it contains two antigen binding sites, but not the site for complement fixation. In the context of the present invention, the F(ab′)₂ fragment may be prepared by gene expression of the relevant DNA sequences.

As used herein, the term “Fv fragment” refers to the N-terminal part of the Fab fragment of an immunoglobulin molecule, consisting of the variable portions of one light chain and one heavy chain. Single-chain Fvs (about 30 KDa) are artificial binding molecules derived from whole antibodies, but which contain the minimal part required to recognise antigen.

In another preferred embodiment, the targeting element is a synthetic or natural peptide, a growth factor, a hormone, a peptide ligand, a carbohydrate or a lipid.

The targeting element can be designed or selected from a combinatorial library to bind with high affinity and specificity to the target antigen. Typical affinities are in the 10⁻⁶ to 10⁻¹⁵ M Kd range. Functional amino acid residues, present in the targeting element, which could participate in the therapeutic agent attachment reaction may be altered by site-directed mutagenesis where possible, without altering the properties of the targeting element. Examples of such changes include mutating any free surface thiol-containing residues (cysteine) to serines or alanines, altering lysines and arginines to asparagines and histidines, and altering serines to alanines.

The target cells themselves can be human, other mammalian cells or microbial cells (e.g. anti-bacterial PDT using anti-bacterial antibodies [Devanathan, S et al. (1990); PNAS (USA) 87, 2980-2984].

In another preferred embodiment, the conjugate of the invention comprises a polypeptide carrier, a compound according to the invention, and optionally, a targeting element.

In one particularly preferred embodiment, the conjugate comprises a polypeptide carrier and a compound according to the invention. In an especially preferred embodiment, the conjugate comprises a polypeptide carrier which comprises at least one alpha-helix having synthetically attached thereto a compound according to the invention. More preferably still, the conjugate comprises at least one alpha-helix having synthetically attached thereto a plurality of porphyrins according to the invention, wherein said porphyrins may be the same or different and are spatially oriented on the polypeptide so as to minimise interactions between said moieties.

As used herein, the term “synthetically attached” encompasses straightforward chemical synthetic techniques and also in vivo synthesis using recombinant DNA techniques.

Preferably, the compounds of the invention are spatially oriented on the polypeptide carrier so as to minimise unfavourable or disruptive interactions between said compounds.

Preferably, the polypeptide carrier of the invention comprises one or more specific amino acid residues for the purpose of site-specific conjugation to said compounds of the invention.

In one preferred embodiment, said specific amino acid residues comprise one or more basic amino acids.

In one preferred embodiment, said specific amino acid residues comprise one or more acidic amino acids.

In another preferred embodiment, said specific amino acid residues comprise one or more hydroxyl-containing amino acids.

In another preferred embodiment, said specific amino acid residues comprise one or more thiol-containing amino acids.

In another preferred embodiment, said specific amino acid residues comprise one or more hydrophobic amino acids. By way of definition, the term “hydrophobic amino acid residue” encompasses amino acids having aliphatic side chains, for example, valine, leucine and isoleucine.

In a particularly preferred embodiment of the invention, the alpha-helix comprises at least two functional amino acid residues positioned so as to protrude externally from said alpha-helix so that each functional amino acid residue does not hinder another. Preferably, the functional amino acid residues are suitable for cross-linking to one or more compounds of the invention. Examples of such functional amino acids include lysine, cysteine, threonine, serine, arginine, glutamate, aspartate, tyrosine.

Typically, the polypeptide may be a conjugate, for example, a protein conjugate, i.e., a fusion protein.

Typically, the α-helix is proteolytically and temperature stable, and is designed so that functional groups from one type of side chain (e.g. basic residues such as lysine and arginine) protrude from the helix in such a way that each functional group is spatially separated from each other.

The length of the helical peptide may be varied to incorporate more or fewer functional amino acid residues, thereby accommodating more or fewer compounds of the invention respectively, as required. Likewise, the position and number of functional amino acid residues can be altered to increase or decrease the distance between the attached porphyrins, or to vary the number of porphyrins attached. In each case, the spatial arrangement of the functional amino acid residues is such that there is little or no interference between the porphyrins attached thereto.

Preferably, the alpha-helix is a 19-residue helix with functional amino acid residues at positions 2, 8, 10, 14 and 16.

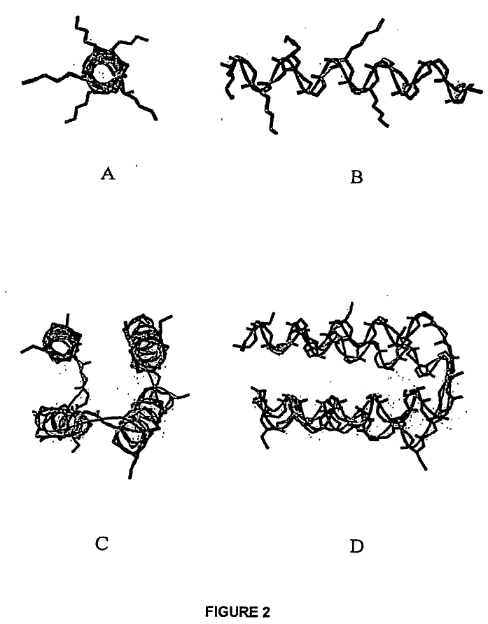

By way of example, and as illustrated in FIG. 2A, the polypeptide carrier may comprise a 19-residue peptide helix with functional amino acids such as lysine or arginine residues at positions 2, 8, 10, 14, 16. This results in an approximately equal number of positively charged residues above/below or either side of the helical axis (viewed in FIG. 2B). These positively charged residues can be seen to be spatially separated when the helix is viewed ‘end on’ (FIG. 2A).

In one preferred embodiment, the polypeptide carrier may comprise two or more alpha-helical polypeptides in the form of a multi-helix bundle. Such multi-helix bundles enable the attachment of a greater number of therapeutic agents. Furthermore, without wishing to be bound by theory, it is believed that multi-helix bundles of this type may exhibit an improved stability over the corresponding single alpha-helical polypeptides.

Thus, in one preferred embodiment, the polypeptide carrier comprises two, three or four alpha-helices, i.e., a two-helix, three helix, or four-helix bundle. Each helix can be of a single-chain or separate chain format.

In one particularly preferred embodiment, the polypeptide carrier further comprises one or more additional amino acid sequences selected from a sub-cellular targeting peptide and a membrane active peptide.

In one preferred embodiment, the sub-cellular targeting peptide targets the nucleus and comprises a sequence selected from KKKKRPR and KRPMNAFIVWSRDQRRK.

In another preferred embodiment, the sub-cellular targeting peptide targets the mitochondria and comprises the sequence MLVHLFRVGIRGGPFP GRLLPPLRFQTFSAVRYSDGYRSSSLLRAVAHLPSQLWA.

In yet another preferred embodiment, the sub-cellular targeting peptide targets lysosomes and comprises the sequence KCPL.

In a further preferred embodiment, the sub-cellular targeting peptide allows proteins to traffic back to the endoplasmic reticulum and comprises the sequence KDEL.

In another preferred embodiment, the membrane active peptide targets the membrane and comprises a sequence selected from the following:

(i)
GLFGAIAGFIENGWEGMIDGWYG;
(ii)
GIEDLISEVAQGALTLVP;
(iii)
ACYCRIPACIAGERRYGTCIYQGRLWAFCC;
and
(iv)
FFGAVIGTIALGVATSAQITAGIALAEAR.

The polypeptide carrier may also comprise a glycosylated protein. For example, the polypeptide may comprise a protein having one or more N- or O-linked carbohydrate residues spatially oriented so as to minimise interactions between said carbohydrates or compounds of formula I attached thereto.

Thus, in one preferred embodiment, the polypeptide carrier comprises a glycosylated protein (e.g. human serum albumin) or comprises a protein having one or more N- or O-linked glycosylation sites. By way of definition, the term “glycosylated protein” refers to a glycoprotein, i.e., a protein having one or more carbohydrates attached thereto. Typically, glycoproteins contain oligosaccharide units linked to either asparagine side chains by N-glycosidic bonds, or to serine and threonine side chains by O-glycosidic bonds. Accordingly, a protein having N- or O-linked glycosylation sites includes any protein containing amino acid residues having one or more OH or NH₂ side chains.

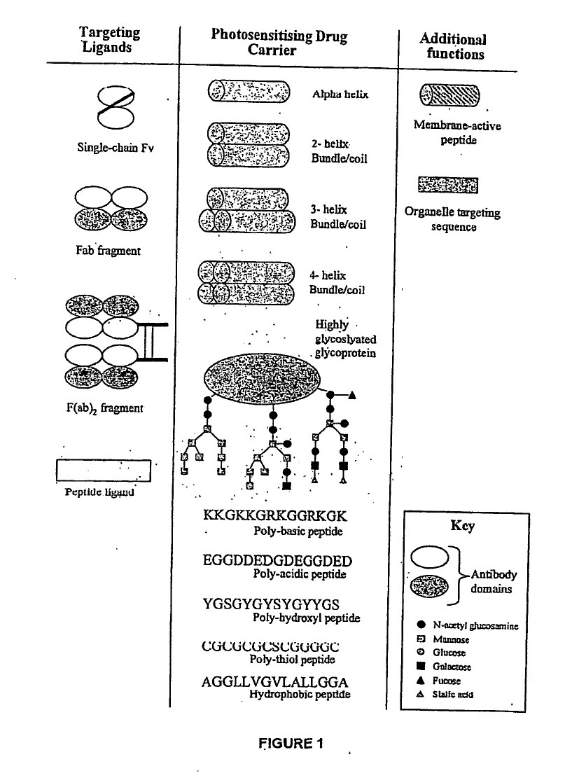

These proteins may be expressed in a eukaryotic system such as mammalian cells, yeasts or insect cells, to ensure full glycosylation. Compounds of the invention whose chemistry is compatible with chemical attachment to hydroxyl or carboxylate groups may be cross-linked onto the glycosylated proteins. The types of carbohydrate residues found on glycosylated proteins are shown in FIG. 1.

In another preferred embodiment of the invention, the polypeptide carrier comprises one or more glycosylation motifs. Typical examples of such glycosylation motifs include Asn-X-Ser and Asn-X-Thr, wherein X is any amino acid residue. Polypeptide sequences including these glycosylation motifs may be expressed in eukaryotic hosts, for example, yeast. Methods for expressing polypeptide sequences may be accomplished by standard procedures well known to those skilled in the art.

After glycosylation, compounds of the invention may be attached to the carbohydrate residues by standard chemical techniques. The spatial arrangement of the glycosylation motifs is such that there is little or no interference between the porphyrins attached thereto.

A further aspect relates to the use of a compound of the invention in the preparation of a conjugate as described above.

Pharmaceutical Compositions

Another aspect of the invention relates to a pharmaceutical composition comprising a compound of the invention, or a conjugate thereof as defined above, admixed with a pharmaceutically acceptable diluent, excipient or carrier.

Even though the compounds/conjugates of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of the present invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes solvate forms of the compounds of the present invention. The terms used in the claims encompass these forms.

Polymorphs

The invention furthermore relates to compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

Therapeutic Uses

A further aspect of the invention relates to the use of a compound/conjugate as described hereinbefore in the preparation of a medicament for treating a proliferative disorder.

In accordance with the invention, there is provided a method for treating a disease or disorder which is characterised by the presence in the body of diseased or undesired cells, such as tumours, viral infections such as HIV, autoimmune disorders such as rheumatoid arthritis or a disease which can be effectively treated by PDT such as age related macular degeneration (AMD).

Preferably, the proliferative disorder is cancer.

As used herein the phrase “preparation of a medicament” includes the use of a compound or conjugate of the invention directly as the medicament in addition to its use in a screening programme for the identification of further agents or in any stage of the manufacture of such a medicament.

Diseases which may be treated according to the invention include cancer, age-related macular degeneration, microbial infections, arthritis and other immune disorders and cardiovascular disease.

Yet another aspect of the invention provides a method of treating a proliferative disorder, said method comprising administering to a subject a therapeutic amount of a compound of the invention, or a conjugate thereof.

Another aspect of the invention relates to the use of a compound/conjugate as described hereinbefore in the preparation of a medicament for photodynamic therapy. More specifically, the compounds of the invention may be used as photodynamic therapeutic (PDT) agents. The combination of a sensitiser and electromagnetic radiation for the treatment of cancer is commonly known as photodynamic therapy. In the photodynamic therapy of cancer, dye compounds are administered to a tumour-bearing subject, these dye substances may be taken up, to a certain extent by the tumour. Upon selective irradiation with an appropriate light source (e.g. a laser) the tumour tissue is destroyed via the dye mediated photo-generation of a species such as singlet oxygen or other cytotoxic species such as free radicals, for example hydroxy or superoxide. This requires the sensitiser to have a high triplet yield and lifetime in order to have the best chance of sensitising singlet oxygen production. It also requires a source of laser illumination into the tumour, the cheapest and most penetrating laser light being red.

Phosphoimmunoassays

Yet another aspect of the invention relates to the use of compounds or conjugates of the invention in phosphoimmunoassays (PIA) and/or in the measurement of dissolved oxygen levels in biological systems.

It known in the art that metalloporphyrins, particularly the Pt and Pd complexes, are potentially useful in phosphoimmunoassays (PIA) [A P Savitsby et al, Dokl. Acad. Nauk SSSR, 1989, 304, 1005]. Furthermore, the extreme sensitivity of the triplet excited states of these metalloporphyrins to dissolved oxygen has been used to measure dissolved O₂ levels in biological systems [T J Green et al, Anal. Biochem., 1988, 174, 73; EP 0127797A and U.S. Pat. No. 4,707,454]. Since the porphyrin derivatives of the present invention exhibit very high triplet yields, they are expected to exhibit improved characteristics with respect to PIA and/or sensitivity to dissolved O₂

Medical Imaging

One aspect of the invention relates to the use of a compound or conjugate as described hereinbefore for medical imaging. By way of example, water soluble paramagnetic manganese complexes of the porphyrins of the invention, including Mn(III) porphyrins, may be used in methods for enhancing images obtained from magnetic resonance imaging of a region containing a malignant tumour growth.

In particular, the chromophores of the invention are capable on excitation of emitting fluorescent light and the presence of a conjugating group enables control over the localisation of the chromophore in vitro and in vivo, making them useful in fluorescence analysis and imaging applications including FACS (Fluorescence Activated Cell sorting).

Other Applications

The compounds of the present invention may be used in a broad range of other applications. By way of example, these may include use as pigments or dyes, as components of discotic liquid crystal phases (in particular they may also be used a precursors for discotic liquid crystals), as two dimensional conjugated polymeric arrays [Drain and Lehn, J. Chem. Soc., Chem. Commun., 1994, 2313]; as reverse saturable absorbers and as molecular wires (R. J. M. Nolte at al, Angew. Chem. Int. Ed. Eng., 1994, 33(21), 2173). The types of liquid crystal devices include linear and non-linear electrical, optical and electro-optical devices, magneto-optical devices and devices providing responses to stimuli such as temperature changes and total or partial pressure changes. The compounds of the present invention may also be used in biaxial nematic devices and as second or third order non-linear optic (NLO) materials.

The compounds of the present invention may be suitable as optical storage media and may be combined with dyes for use in laser addressed systems, for example in optical recording media. Typically the porphyrin will absorb in the near-infrared. In order to make an optical recording media using a near-infrared absorber, the near-infrared absorber may be coated or vacuum-deposited onto a transparent substrate. EP 0337209 A2 describes the processes by which the above optical-recording media may be made. The compounds of the present invention are also useful in near-infrared absorption filters and liquid crystal display devices. As described in EP 0337209 A2, display materials can be made by mixing a near-infrared absorber of the invention with liquid crystal materials such as nematic liquid crystals, smectic liquid crystals and cholesteric liquid crystals. The compounds of the present invention may be incorporated into liquid crystal panels wherein the near infrared-absorber is incorporated with the liquid crystal and a laser beam is used to write an image. Mixtures of porphyrins of the current invention may be mixed with liquid crystal materials in order to be used in guest-host systems. GB 2,229,190 B describes the use of phthalocyanines incorporated into liquid crystal materials and their subsequent use in electro-optical devices.

It may also be advantageous to polymerise certain of the compounds of the current invention. There are numerous ways in which the porphyrins may be incorporated into a polymer. Polymerisation may be effected by one or more of the positions R₁-R6 in formula I, or via the central metal atom or metal compound, or by a combination of the above techniques.

Polymerised porphyrins may also be used in Langmuir Blodgett films. Langmuir Blodgett films incorporating porphyrins of the present invention may be laid down using conventional and well known techniques, see R. H. Tredgold in “Order in Thin Organic Films”, Cambridge University Press, p 74, 1994 and reference therein. Langmuir Blodgett Films incorporating compounds of the present invention may be used as optical or thermally addressable storage media.

Synthesis

Another aspect of the invention relates to a process for preparing compounds of formula I, said process comprising reacting a compound of formula II with a dipyrrole of formula III to form a compound of formula Ib, in which R₁ is H, and X, R₂ are as defined hereinbefore,

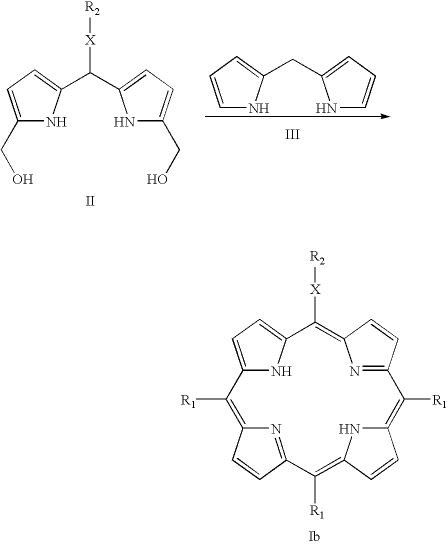

Preferably, dialcohol II is obtained by reducing the corresponding dialdehyde precursor, for example, by treating with sodium borohydride. The dialdehyde itself can be obtained by treating the alpha-unsubstituted dipyrrole with POCl₃/DMF and NaOH.

Preferably, the process of the invention further comprises the step of converting said compound of formula Ib to a compound of formula Ic in which R₁ is halogen, X, R₂ are as defined above.

In one particularly preferred embodiment, the compound of formula Ib is treated with (CF₃CO₂)₂PhI in a suitable solvent system, for example, a CHCl₃/pyridine mixture.

Even more preferably, the process further comprises the step of reacting the compound of formula Ic with 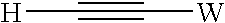
to form a compound of formula Id wherein R₁ is 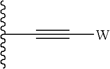
and X, R₂ and W are defined hereinbefore.

In one especially preferred embodiment of the invention, the conversion of the compound of formula Ic to Id is achieved using 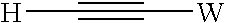
and tetrakis-triphenylphosphine palladium (0) in a mixture of tetrahydrofuran and triethylamine.

By way of example, in one particularly preferred embodiment of the invention, the compound of formula I is synthesised in accordance with Scheme 1, shown below.

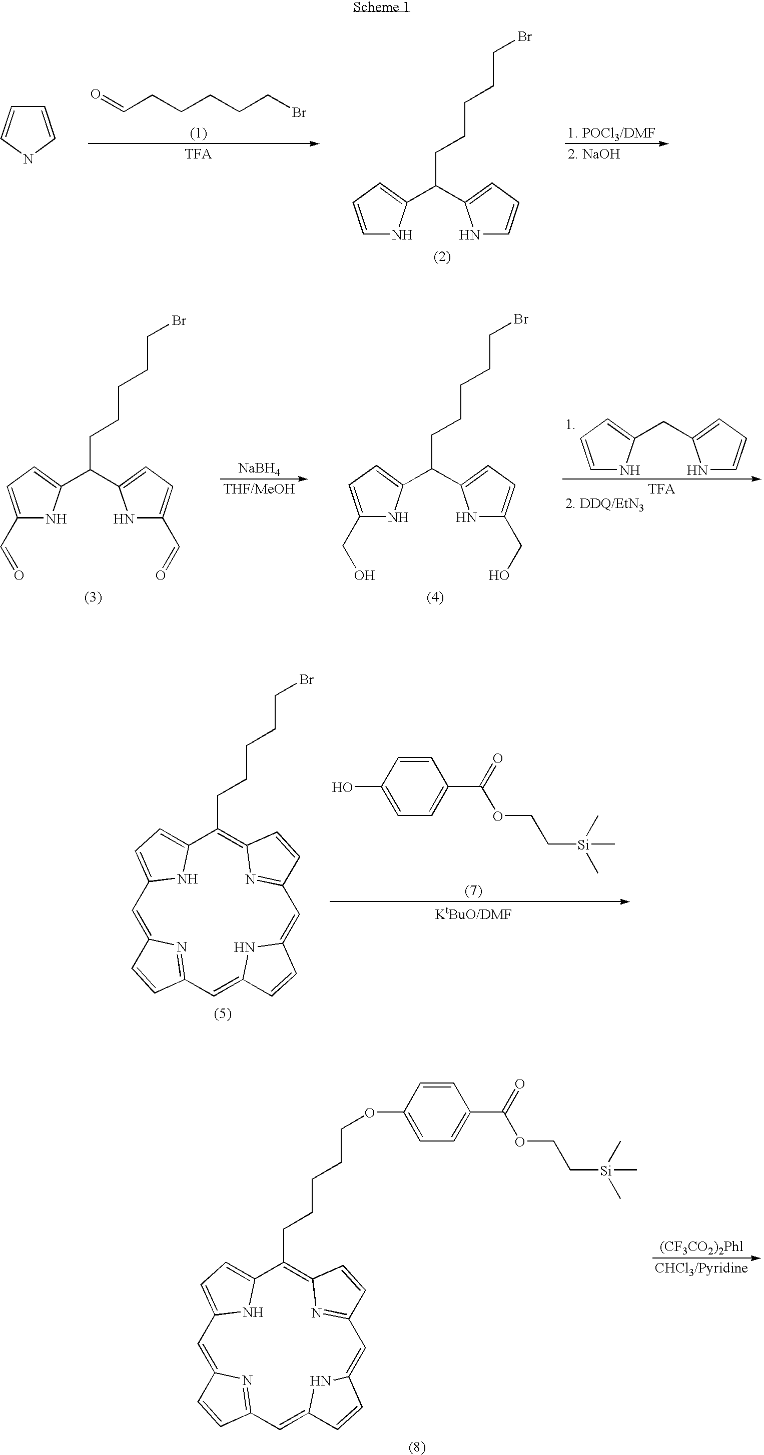

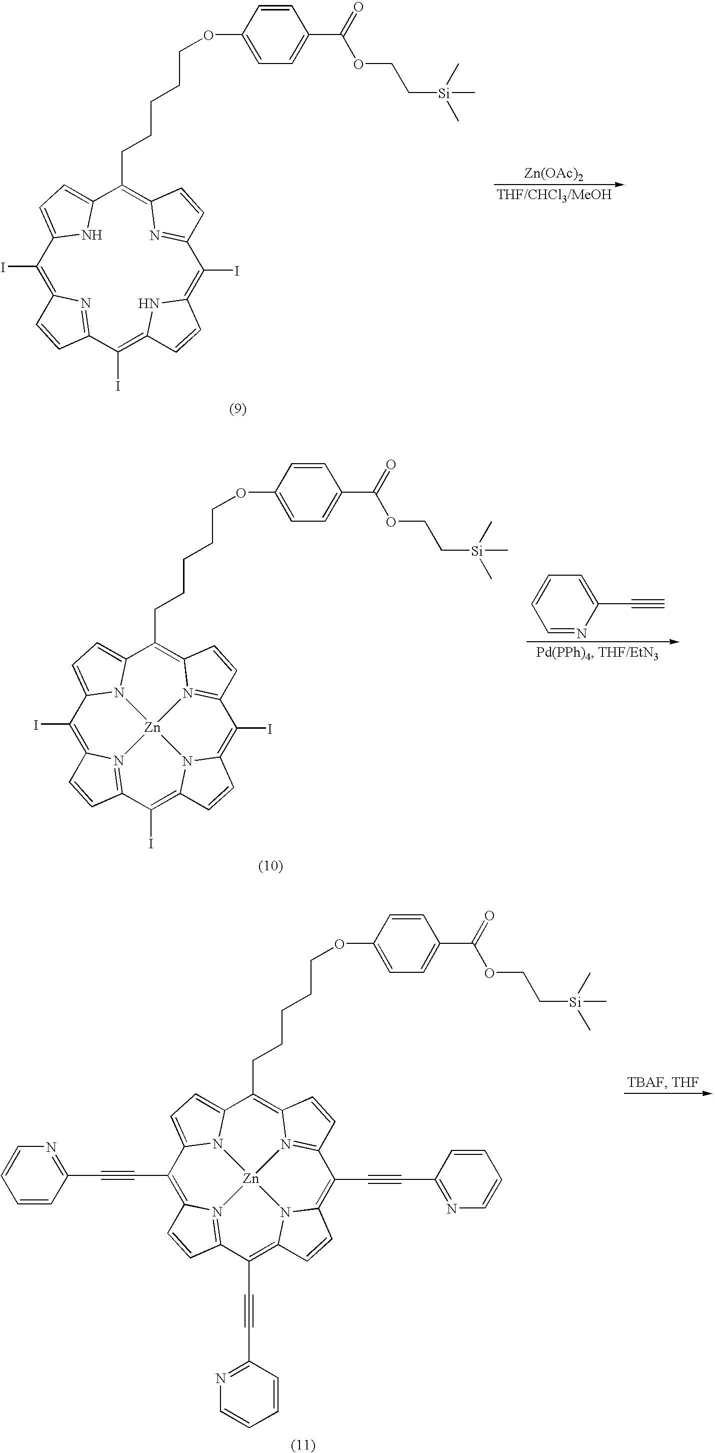

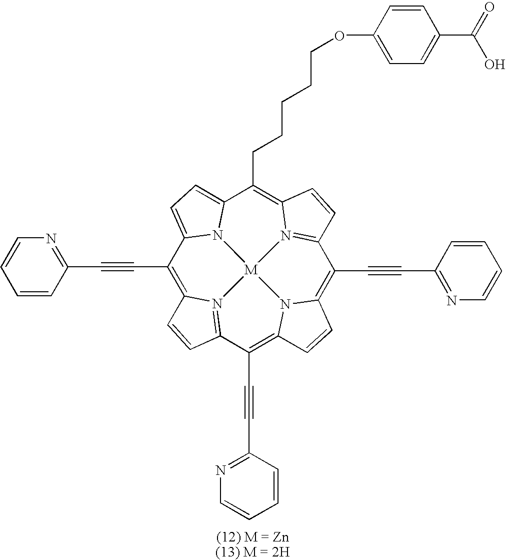

The present invention is further described by way of example, and with reference to the following figures wherein:

FIG. 1 shows the modular structure of a multifunctional targetable-carrier protein.

FIG. 2 shows the molecular structure of helical based carrier proteins for the porphyrins described herein. In more detail, FIGS. 2(A) and (B) show a single peptide α-helix engineered to contain optimally-spaced lysine or arginine residues, which can be used to deliver porphyrins. Side (B) and end-on (A) views show favourable spacing of the amino groups used to attach the porphyrins. FIGS. 2(C) and (D) show a 4-helix bundle, engineered to contain optimally-spaced cysteine residues, which can be used to deliver porphyrins. Side (B) and end-on (A) views show favourable spacing of the thiol groups used to attach the porphyrins.

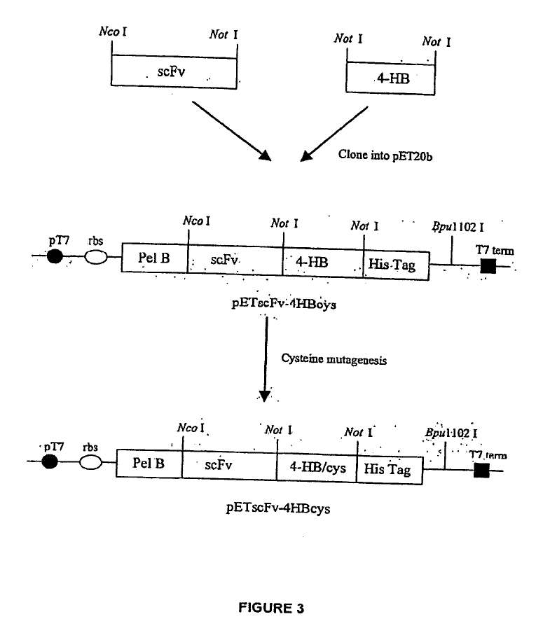

FIG. 3 shows the construction of an scFv-4-helix bundle fusion gene. In more detail, FIG. 3 shows how a scFv and a 4-helix bundle gene would be assembled in a bacterial expression vector to produce the scFv-helix bundle fusion protein.

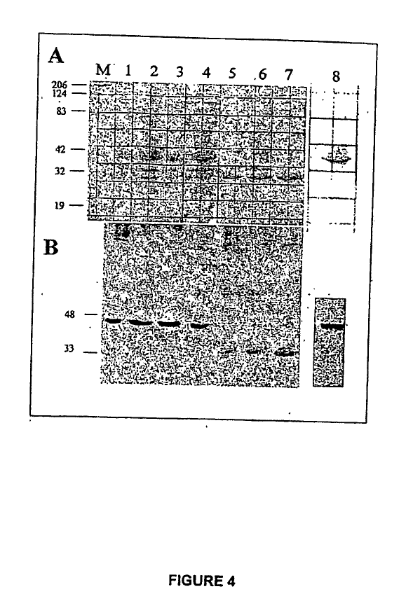

FIG. 4 shows over-expression anti-CEA scFv (lanes 5-7) and scFv-4 helix bundle (lanes 1-4) fusion protein in E. coli BL21(DE3). (A) Whole cell lysates are analysed by SDS-PAGE stained with coomassie blue. (B) Whole cell lysates are analysed by western blot using a mouse anti-His tag monoclonal antibody (Qiagen) followed by anti mouse-horseradish peroxidase (Sigma) developed by ECL (Amersham). M-molecular weight markers in KDa. Lane 8 represents substantially pure scFv-4 helix bundle fusion protein after IMAC on Nickel sepharose.

FIG. 5 shows the absorption spectrum of porphyrin [12].

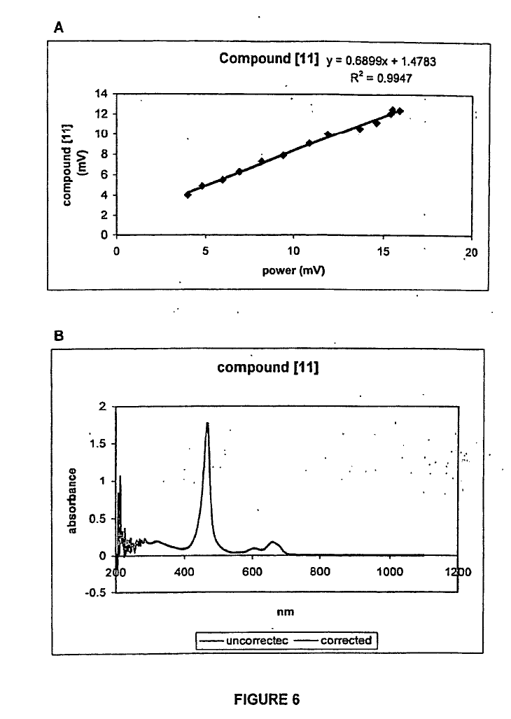

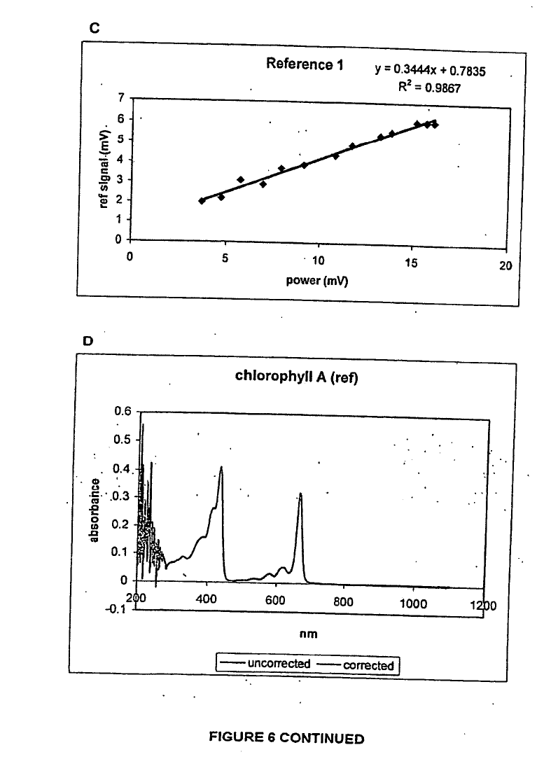

FIG. 6A shows a plot of mV against power (mV) for compound [11]; FIG. 6B shows the UV-visible spectrum (absorbance versus wavelength) for compound [11]; FIG. 6C shows a plot of mV against power (mV) for reference 1 (chlorophyll A); FIG. 6D shows the UV-visible spectrum (absorbance versus wavelength) for chorophyll A.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### EXAMPLES

### 6-Bromohexanal [1]

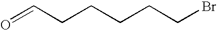

Ref: Liebigs. Ann. Chem., 1991, 569

In a round bottomed flask fitted with a reflux condenser, 8.63 g (40.02 mmol) of pyridinium chlorochromate (PCC) was suspended in DCM (60 ml) and the mixture brought to reflux. At this point a solution of 6-bromohexanol (4.31 g, 26.68 mmol) in DCM (5 ml) was added in one portion to the stirred suspension. After 1.5 h, 50 ml of diethyl ether was added and the supernant liquid decanted from the black gum. The insoluble residue was washed several times with diethyl ether. The combined organic washings was passed through a pad of silica gel, eluting with DCM and concentrated to give a light green oil (93%). To minimise decomposition, the crude aldehyde was used in the next step without purification.

### 5-(6-Bromohexane)dipyrromethane [2]

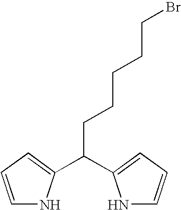

Ref: J. Mater. Chem., 2001, 1162

6-Bromohexanal [1] (3.5 g, 19.54 mmol) was dissolved in pyrrole (33.9 ml, 48.86 mmol) and the resulting solution degassed with argon for 5 min before the addition of TFA (0.150 ml, 1.95 mmol). The reaction was stirred for 15 min under argon at room temperature then quenched by the addition of NaOH 0.1M (50 ml). The reaction mixture was then extracted with ethyl acetate (2×50 ml), the combined organic extracts washed with water, dried (MgSO₄), and concentrated under vacuum to give a brown oil. This was purified by column chromatography [silica gel, hexane/ethyl acetate/triethyl amine (60:40:1)], to give the dipyrromethane as a yellow oil (83%). ¹H NMR δ (ppm, CDCl₃): 7.26 (br s, 2H, NH);6.63 (m, 2H, pyrrole α-H); 6.158, 6.151, 6.144, 6.137 (q, 2H, pyrrole β-H, J=2.9 Hz); 6.07 (br s, 2H, pyrrole β′-H); 3.988, 3.969, 3.950 (t, 1H, methane bridge H, J=7.6 Hz); 3.398, 3.381, 3.364 (t, 2H, —CH₂CH₂Br, J=6.7 Hz); 1.98-1.92 (m, 2H, —CH₂(CH₂)₄Br); 1.85-1.80 (m, 2H, —CH₂CH₂Br); 1.48-1.42 (m, 2H, —CH₂(CH₂)₃Br); 1.34-1.31 (m, 2H, —CH₂(CH₂)₂Br). ¹³C NMR δ (ppm, CDCl₃): 133.33 (pyrrole γ-C); 117.08 (pyrrole α-C); 108.03 (pyrrole β-C); 105.41 (pyrrole β′-C); 37.50 (methane bridge-C); 34.30, 33.94, 32.61, 27.96, 26.68 (alkyl chain). MS (EI⁺) m/z 294 (M⁺ 6%), 296 (M⁺ 6%), 145 (100); found 294.07295 (calculated for C₁₄H₁₉BrN₂ 294.07316).

### 1,9-Bisformyl-5-(6-bromohexane)dipyrromethane [3]

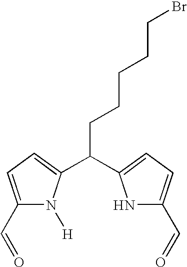

The formylation was carried out using a large excess of the Vilsmeier reagent prepared using freshly distilled POCl₃ (3.2 ml), which was added dropwise at 0° C. to 22 ml of anydrous DMF. The dipyrromethane [2] (3.0 g, 9.70 mmol) was dissolved in anhydrous DMF (18 ml) and cooled down to 0° C., 22 ml of the previously prepared Vilsmeier reagent was then added dropwise. The reaction mixture was stirred under argon at 0° C. for 2 h, allowed to warm to room temperature at which point the iminium salt was hydrolysed by the addition of 1M KOH solution (until pH>10). The resulting dark brown oil was extracted with ethyl acetate (2×100 ml), the combined organic layers washed with water, dried (Na₂SO₄), filtered and concentrated. The crude oil was purified column chromatography [silica gel chloroform/ethyl acetate (8:2)] to give a brown oil (63%). ¹H NMR δ (ppm, CDCl₃): 11.33 (br s, 2H, NH); 9.44 (s, 2H, —CHO); 6.943, 6.941, 6.935 (m, 2H, pyrrole-β H); 6.208, 6.206, 6.199 (m, 2H, pyrrole-β′ H); 4.235, 4.215, 4.195 (t, 1H, methane bridge H, J=7.9 Hz); 3.378, 3.362, 3.345 (t, 2H, —CH₂CH₂Br, J=6.7 Hz); 2.20-2.14 (m, 2H, —CH₂(CH₂)₄Br); 1.86-1.79 (m, 2H, —CH₂CH₂Br); 1.51-1.44 (m, 2H, —CH₂(CH₂)₃Br); 1.37-1.31 (m, 2H, —CH₂(CH₂)₂Br). ¹³C NMR δ (ppm, CDCl₃): 179.42 (—CHO); 142.91 (pyrrole γ-C); 132.66 (pyrrole α-C); 123.40 (pyrrole β-C); 109.63 (pyrrole β′-C); 38.42 (methane bridge-C); 33.66, 33.12, 32.37, 27.72, 26.81 (alkyl chain); MS (EI⁺) m/z 350 (M⁺ 8%), 201 (100), 145 (45); found 350.06282 (calculated for C₁₆H₁₉N₂O₂Br 350.06299.

### 1,9-Bishydroxymethyl-5-(6-bromohexane)dipyrromethane [4]

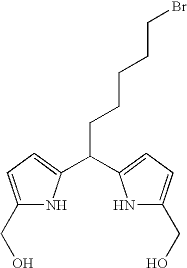

The bisformyl-dipyrromethane [3] (1.0 g, 2.74 mmol) was dissolved in a mixture of THF/methanol (3:1, 137 ml). NaBH₄ (5.17 g, 0.137 mol) was then added in small portions and its progressed followed by TLC [neutral alumina oxide, DCM/5% methanol]. The reaction was stirred at room temperature, the colour changing to a light yellow, after 30 min it was quenched by the addition of water (CARE!). The reaction mixture was extracted in diethyl ether (2×100 ml) and the combined organic layers washed with a saturated solution of Na₂CO₃, dried (Na₂SO₄), filtered and concentrated to give a light yellow solid (96%). ¹H NMR δ (ppm, CDCl₃): 9.89 (br s, 2H, NH); 5.935, 5.930 (d, 4H, pyrrole β and β′ H, J=2.3 Hz); 4.28 (s, 4H, —CH₂OH); 3.889, 3.870, 3.851 (t, 1H, methane bridge H, J=7.5 Hz); 3.376, 3.359, 3.342 (t, 2H, —CH₂CH₂Br, J=6.7 Hz); 1.89-1.78 (m, 4H, CH₂CH₂Br and —CH₂(CH₂)₄Br); 1.46-1.39 (m, 2H, —CH₂(CH₂)₃Br); 1.30-1.21 (m, 2H, —CH₂(CH₂)₂Br). ¹³C NMR δ (ppm, CDCl₃): 135.50 (pyrrole α-C); 131.85 (pyrrole γ-C); 105.78 (pyrrole β-C); 105.13 (pyrrole β′-C); 57.62 (—CH₂OH); 37.41 (methane bridge C); 33.93, 33.28, 32.68, 27.92, 26.46 (alkyl chain). MS (FAB) m/z 355 (M⁺ 15%), 354 (M⁺ 15%), 337 (27%), 260 (24%), 205 (100); found 354.09429 (calculated for C₁₆H₂₃BrN₂O₂354.09402)

### 5-(5-Bromopentane)porphyrin [15]

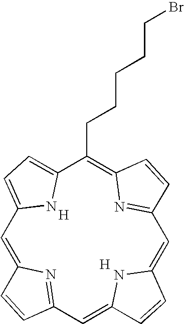

To a light protected stirred solution of the dipyrromethanediol [4] (0.2 g, 0.56 mmol) and the unsubstituted dipyrromethane (0.082 g, 0.56 mmol) in CH₃CN (200 ml), under argon, TFA was added (0.52 ml, 6.7 mmol) and the reaction stirred at room temperature for 1 h. DDQ (0.38 g, 1.68 mmol) was then added to oxidise the resulting porphyrinogen and the mixture stirred open to the air at room temperature for another 1 hr. The resulting mixture was neutralized by the addition of triethyl amine (0.94 ml, 6.7 mmol) and the solution filtered through a large pad of silica, eluting with DCM. The front running porphyrinic red band was collected and purified column chromatography [silica gel, chloroform/hexane (6:4)] to give the porphyrin [7] as a red solid (16%). ¹H NMR δ (ppm, CDCl₃): 10.18 (s, 2H, meso-H attached to C₁₀ and C₂₀); 10.10 (s, 1H, meso-H attached to C₁₅); 9.55, 9.54 (d, 2H, pyrrole-H at C₃ and C₇, J=4.6 Hz); 9.42-9.36 (m, 6H, pyrrole-H); 5.016, 4.996, 4.976 (t, 2H, meso-CH₂, J=8.0 Hz); 3.438, 3.422, 3.405 (t, 2H, J=6.5 Hz, —CH₂Br); 2.57-2.49 (m, 2H, —CH₂(CH₂)₃Br); 2.03-1.98 (m, 2H, —CH₂(CH₂)₂Br); 1.94-1.87 (m, 2H, —CH₂CH₂Br); −3.66 (br s, 2H, NH). ¹³C NMR δ (ppm, CDCl₃): 146.79, 146.44, 145.02, 144.73 (two broad doublets); 131.80, 131.63, 130.83, 128.00, 119.19, 104.20, 102.85 (rest of C at the porphyrin ring); 34.80, 33.75, 32.77, 29.73, 29.00 (alkyl chain). MS (FAB⁺) 461 (M⁺ 35%), 459 (M⁺ 37%); found 459.11752 (calculated for C₂₅H₂₃BrN₄ 459.11843; UV-Vis(DCM) λ₍ₘₐₓ₎ 398, 497, 569 nm.

### 2′-(trimethylsilyl)ethyl-4-acetoxybenzoate [6]

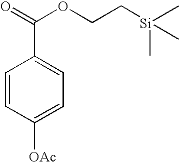

Ref: J. Med. Chem., 1998, 41, 3062

4-acetoxybenzoic acid (1.62 g, 9 mmol) was suspended in acetonitrile (4 ml), then tetrachloromethane (2 ml) was added and the mixture was stirred vigorously for 5 minutes. During this time the suspension liquified and became completely transparent. Stirring was continued for 15 min at which point trimethylsilylethanol (1.16 g, 10 mmol) was added and the reaction refluxed for 15 min. Upon cooling, the reaction mixture was poured into stirring diethyl ether (200 ml), filtered, dried (MgSO₄) and the solvent removed. The reaction crude was purified by column chromatography [silica gel, hexane:ethylacetate (7:3)] to give [6] as a colourless oil (60%). ¹H NMR δ (ppm, CDCl₃): 8.077, 8.05, 7.172, 7.151 (dd, 4H, phenyl-H, J=8.7 Hz), 4.43-4.39 (m, 2H, —OCH₂CH₂Si(CH₃)₃); 2.32 (s, 3H, Ph-OCOCH₃); 1.15-1.10 (m, 2H, —OCH₂CH₂Si(CH₃)₃); 0.079 (s, 9H, —Si(CH₃)₃). ¹³C NMR δ (ppm, CDCl₃): 168.92 (Ph-OCOCH₃); 165.96 (Ph-OCO(CH₂)₂Si(CH₃)₃); 154.11 (C₄ benzene ring); 131.05 (C₂ and C₆ benzene ring); 128.22 (C₁ benzene ring); 121.52 (C₃ and C₅ benzene ring); 63.38 (—OCH₂CH₂Si(CH₃)₃); 21.16 (Ph-OCOCH₃); 17.38 (—OCH₂CH₂Si(CH₃)₃); −1.45 (—OCH₂CH₂Si(CH₃)₃). MS (EI): 279 (M⁺, 2%), 195(100%), 121(52), 73 (47); found 279.10545 (calculated for C₁₄H₂₀O₄Si 279.10526).

### 2′-(trimethylsilyl)ethyl-4-hydroxybenzoate [7]

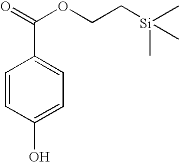

Ref: J. Med. Chem., 1998, 41, 3062

An aqueous solution of sodium carbonate (1%, 19 ml) was added slowly to a solution of [6] in THF/MeOH (5:5, 12 ml). The resulting mixture was stirred at room temperature and the reaction monitored by TLC [silica, hexane/ethylacetate (7:3)]. On completion, the THF and MeOH were removed in vacuo and the remaining aqueous phase was extracted with diethyl ether (2×50 ml). The combined organic phase was dried over MgSO₄, filtered, the solvent removed and a thick oil crystallized under high vacuum to yield [7] as a white solid (88%). ¹H NMR δ (ppm, CDCl₃): 7.955, 7.933, 6.897, 6.875 (dd, 4H, phenyl-H, J=8.7 Hz); 5.29 (s, 1H, Ph-OH); 4.42-4.38 (m, 2H, OCH₂CH₂Si(CH₃)₃); 1.14-1.10 (m, 2H, —OCH₂CH₂Si(CH₃)₃); 0.068 (s, 9H, —Si(CH₃)₃). ¹³C NMR δ (ppm, CDCl₃): 167.23 (Ph-OCO(CH₂)₂Si(CH₃)₃); 160.29 (C₄ benzene ring); 131.84 (C₂ and C₆ benzene ring); 122.60 (C₁ benzene ring); 115.25 (C₃ and C₅ benzene ring); 63.27 (OCH₂CH₂Si(CH₃)₃); 17.37 (—OCH₂CH₂Si(CH₃)₃); −1.45 (—OCH₂CH₂Si(CH₃)₃). MS (EI): 238 (M⁺, 2%), 195(100%), 121(63), 73 (53); found 238.10198 (calculated for C₁₂H₁₈O₃Si 238.10252).

### 5-[5-(2′-(trimethylsilyl)ethyl-4-hydroxybenzoate)pentane]porphyrin [8]

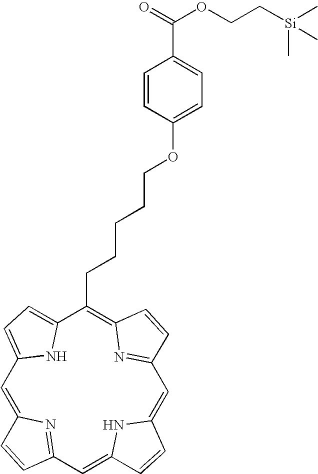

The hydroxybenzoate [7] (51 mg, 0.22 mmol) was dissolved in anhdrous DMF (5 ml), then potassium tert-butoxide (26 mg, 0.23 mmol) was added and the resulting mixture stirred for 5 min before the addition of porphyrin [5] (90 mg, 0.20 mmol). The reaction was sitirred at room temperature, under argon and it was monitored by TLC [silica, CHCl₃/hexane (7:3)]. The reaction was complete within 2 hour, it was then quenched by the addition of water and extracted with diethyl ether (2×100 ml). The combined organic extracts was dried over Na₂SO₄, filtered and concentrated. The crude was purified by column chromatography [silica gel, CHCl₃/hexane (7:3)] to give the porphyrin in good yields (64%). ¹H NMR δ (ppm, CDCl₃): 10.22 (s, 2H, meso-H attached to C₁₀ and C₂₀); 10.13 (s, 1H, meso-H attached to C₁₅); 9.614, 9.606 (d, 2H, pyrrole-H at C₃ and C₇, J=3.5 Hz), 9.44-9.39 (m, 6H, pyrrole-H), 7.985, 7.963, 6.879, 6.857 (dd, 4H, phenyl-H, J=8.7 Hz); 5.09-5.05 (m, 2H,meso-CH₂); 4.43-4.39 (m, 2H, CO₂CH₂CH₂Si(CH₃)₃); 3.97 (m, 2H,O—CH₂); 2.61 (br s, 2H, —CH₂); 1.94 (br s, 4H, —CH₂); 1.16-1.12 (t, 2H, J=4.1 Hz, CO₂CH₂CH₂Si(CH₃)₃); 0.101 (s, 9H, Si(CH₃)₃); −3.59 (br s, 2H, NH). ¹³C NMR δ (ppm, CDCl₃): 166.61 (CO); 162.71 (C₄ benzene ring); 146.82, 146.51, 144.79, 144.21 (two broad doublets); 131.84, 131.66 (porphyrin C); 131.46 (C₂ and C₆ benzene ring); 130.86, 128.12 (porphyrin C);122.89 (C₁ benzene ring); 119.41 (porphyrin C); 114.00 (C₃ and C₅ benzene ring); 104.23, 102.86 (porphyrin C); 67.97 (alkyl chain); 62.87 (OCH₂CH₂TMS); 38.32, 34.91, 29.21, 26.89 (alkyl chain); 17.44 (OCH₂CH₂TMS); −1.39 (—Si(CH₃)₃). MS (FAB⁺) 618 (41), 617 (M⁺ 100%), 616 (76), 323 (41), 73 (42); found 617.29405 (calculated for C₃₇H₄₀N₄O₃Si 617.29479); UV-Vis(DCM) λ₍ₘₐₓ₎ 399, 497, 569 nm.

### 5-[5-(2′-(trimethylsilyl)ethyl-4-hydroxybenzoate)pentane]-10,15,20-triiodo-porphyrin [9]

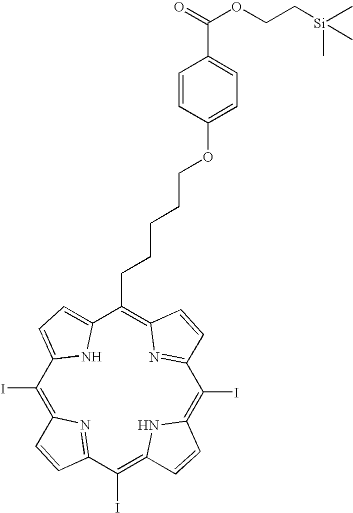

To a solution of (CF₃CO₂)₂PhI (59 mg, 0.136 mmol) in dry CHCl₃ (10 ml) a solution of iodine (29 mg, 0.114 mmol; in 6 ml of dry chloroform) was added followed by a few drops of pyridine. The red-violet solution was sitirred (shielded from light) at room temperature and under argon until it became yellow (about 15 min). This mixture was then added dropwise to a stirred solution of [8] (35 mg, 0.057 mmol) in dry CHCl₃ (30 ml). The reaction was stirred at room temperature, under argon and in the dark. 24 hours latter TLC [silica gel, CHCl₃/hexane (8:2)] showed two spots corresponding to the di- and tri-iodinated porphyrins (R_{f}=0.675 and 0.750 respectively) and more of the iodinating reagent was added [0.068 mmol of (CF₃CO₂)₂PhI and 0.057 mmol of I₂]. The mixture was stirred for a further 24 h and on completion it was quenched by washing it with a saturated solution of sodium thiosulphate (2×100 ml). The chloroform layer was separated, dried (Na₂SO₄) and the solvent removed. The resulting material was purified by column chromatography [silica gel, CHCl₃/hexane (8:2)] to give [9] as a dark solid (47%). MS(FAB) 995(M⁺ 17%), 869 (11); UV-Vis(THF) λ₍ₘₐₓ₎ 430, 527, 575, 612, 673. Due to its limited solubility, the triiodoporphyrin [9] was not fully characterised.

### Zinc5-[5-(2′-(trimethylsilyl)ethyl-4-hydroxybenzoate)pentane]-10,15,20-triiodo-porphyrin [10]

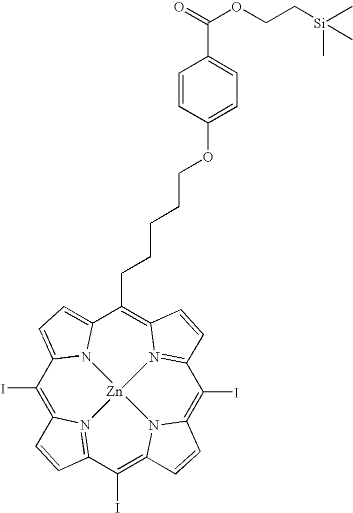

To a solution of porphyrin [9] (43 mg, 0.044 mmol) in THF/CHCl₃/MeOH (17 ml, 25:5:4), zinc acetate (59 mg, 0.269 mmol) was added and the mixture was refluxed for 2 hours. The reaction can be monitored with UV and within 30 min the initially purple solution became green. Then, the reaction was cooled to room temperature and the volume was reduced. The solid obtained was washed with methanol to remove the excess of zinc acetate and filtered to give the metallated porphyrin [10] as a purple solid (89%). ¹H NMR δ (ppm, THF): 9.61-9.59 (m, 6H, pyrrole H at C₂, C₃, C₇, C₈, C₁₂, C₁₃, C₁₇, C₁₈); 9.421, 9.409 (d, 2H, pyrrole H at C₃ and C₇ J=4.7 Hz); 7.887, 7.684, 6.888, 6.866 (dd, 4H, phenyl-H, J=8.8 Hz); 4.87-4.84 (m, 2H, meso-CH₂); 4.33-4.29 (m, 2H, —CO₂CH₂CH₂Si(CH₃)₃); 4.02-4.00 (m, 2H,O—CH₂); 2.45 (m, 2H,—CH₂); 1.91 (m, 4H,—CH₂); 1.08-1.04 (m, 2H, CO₂CH₂CH₂Si(CH₃)₃); 0.038 (s, 9H, —Si(CH₃)₃). ¹³C NMR δ (ppm, THF): 166.25 (CO); 163.83; 154.28, 153.89, 153.03, 152.57 (two doublets, C at porphyrin ring); 139.99, 139.70, 139.15, 132.04, 131.53, 124.48, 123.95, 114.75; 67.97 (alkyl chain); 62.94 (OCH₂CH₂TMS); 39.94, 36.05, 30.24, 27.62 (alkyl chain); 18.13 (OCH₂CH₂TMS); −1.37 (—Si(CH₃)₃); MS(FAB⁺) 1056(M⁺ 2%); found 1055.88659 (calculated for C₃₇H₃₅I₃N₄O₃SiZn 1055.890421); UV-Vis (THF) λ₍ₘₐₓ₎ 435, 573, 621 nm

### Zinc5-[5-(2′-(trimethylsilyl)ethyl-4-hydroxybenzoate)pentane]-10,15,20-tri-2-ethynylpyridine-porphyrin [11]

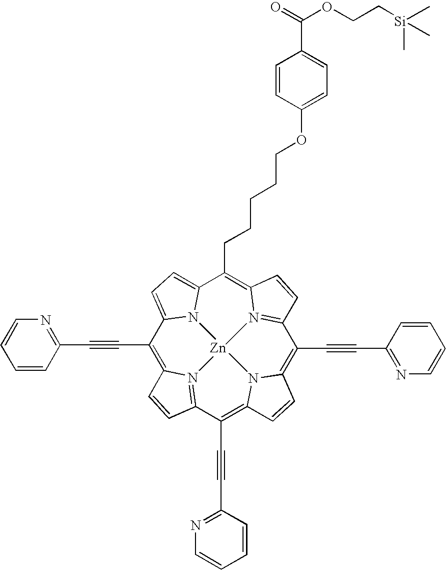

To a solution of the metallated triiodinated porphyrin [10] (20 mg, 0.019 mmol) in dry THF (6.78 ml), dry triethylamine (67.8 μl) was added followed by tetrakis(triphenylphosphine)palladium(0) catalyst (3.4 mg, 0.0029 mmol) and copper (I) iodide (1.35 mg, 0.0071 mmol). Then, 2-ethynylpyridine was added (9.8 mg, 0.095 mmol) and the reaction mixture stirred at room temperature, under argon and shielded from light. The reaction was followed by UV spectroscopy and was complete within 24 h. The resulting dark green mixture was passed through a short pad of silica, eluting with THF and an intense green band was collected. The solvent was removed and the crude purified on a preparative-plate [silica gel, THF/hexane (6:4)] to yield the triacetylatedporphyrin [11] as a green solid (57%). ¹H NMR (δ ppm, THF) 9.77-9.68 (m, 6H, pyrrole-H); 9.45 (d, 2H, J=4.7 Hz, pyrrole-H); 8.84 (m, 2H, pyridyl-H); 8.12 (d, 2H, J=7.67 Hz, pyridyl-H); 7.97-7.91 (m, 5H, pyridyl-H and aryl-H); 7.45 (m, 3H, pyridyl-H); 6.93 (d, 2H, J=8.8 Hz, aryl-H); 4.90 (m, 2H, meso-CH₂); 4.31 (t, 2H, J=8.2 Hz, CO₂CH₂CH₂Si(CH₃)₃); 4.09 (m, 2H,O—CH₂); 2.49 (m, 2H, —CH₂); 1.97 (m, 4H—CH₂); 1.07 (t, 2H, J=8.2 Hz, CO₂CH₂CH₂Si(CH₃)₃); 0.08 (s, 9H, —Si(CH₃)₃); C₅₈H₄₇N₇O₃SiZn calc. MS(FAB⁺) 983(M+2 2%), found 981.279709 (calculated for C₅₈H₄₇N₇O₃SiZn 981.28011; UV-Vis(THF) λ₍ₘₐₓ₎ 466, 607, 668 nm

### Zinc5-[5-(2′-(trimethylsilyl)ethyl-4-hydroxycarboxyphenyl)pentane]-10,15,20-tri-2-ethynylpyridine-porphyrin [12]

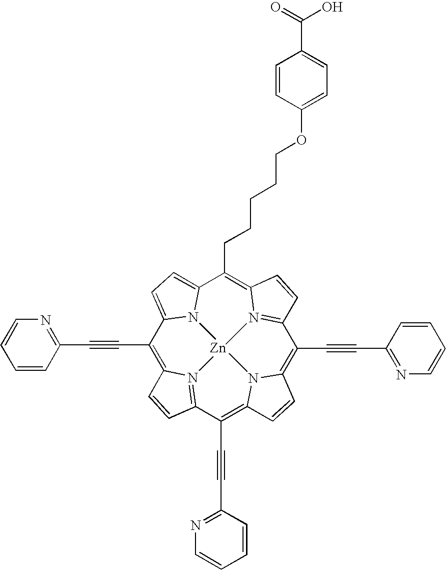

To a light protected solution of porphyrin [11] (20 mg, 0.020 mmol) in dry THF (3 ml), tetrabutylammonium fluoride TBAF (160 μl, 1M solution in THF) was added and the reaction stirred for 4 hours at room temperature, under argon. On completion (as judged by the consumption of starting porphyrin) the reaction mixture was concentrated and purified was by column chromatography on silica gel. Unreacted porphyrin [13] was recovered on eluting with THF/hexane (8:2), then by changing the eluant to CHCl₃/10% MeOH the desired deprotected porphyrin was isolated as a green solid. The solid was washed with 5% NaHCO₃ in water, to remove traces of TBAF, filtered and dried to give porphyrin [12] as a green solid (63%). UV-Vis(MeOH) λ₍ₘₐₓ₎ 466, 610, 668 nm. The absorption spectrum of compound [12] is shown in FIG. 5. An expanded region of the spectrum is inset.

### 5-[5-(2′-(trimethylsilyl)ethyl-4-hydroxycarboxyphenyl)pentane]-10,15,20-tri-2-ethynylpyridine-porphyrin [13]

To a light protected solution of the porphyrin [12] (5 mg) in chloroform TFA (50 μl) was added and the reaction mixture stirred at room temperature under argon. The reaction was followed by UV/visible spectroscopy and was complete in 1 h. UV-Vis(MeOH) λₘₐₓ 455, 570, 690 nm.

### 5-Nonyldipyrromethane [14]

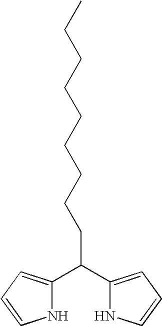

Decylaldehyde (5 g, 0.032 mol) was dissolved in pyrrole (55.5 ml, 0.8 mol) and the resulting solution degassed with argon for 5 min before the addition of TFA (0.246 ml, 3.2 mmol). The reaction was stirred for 10 min under argon at room temperature then quenched by the addition of NaOH 0.1M (50 ml). The reaction mixture was then extracted with ethyl acetate (2×50 ml), the combined organic extracts washed with water, dried (Na₂SO₄), and concentrated. The excesss of pyrrole was removed under vacuum to give a brown oil. This was purified by column chromatography [silica gel: hexane/ethyl acetate/triethyl amine) (80:20:1)], to yield the dipyrromethane as a yellow oil (75%). ¹H NMR δ (ppm, CDCl₃): 7.77 (br s, 2H, NH);6.62 (m, 2H, pyrrole α-H); 6.13 (q, 2H, pyrrole β-H, J=2.97 Hz); 6.05 (m, 2H, pyrrole β′-H); 3.95 (t, 1H, methane bridge H, J=7.55 Hz); 1.22 (m, 16H, alkyl chain); 0.86 (m, 3H, —CH₃).

### 1,9-Bisformyl-5-nonyldipyrromethane [15]

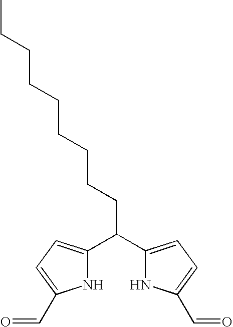

The formylation was carried out using a large excess of the Vilsmeier reagent prepared using freshly distilled POCl₃ (3.6 ml), which was added dropwise at 0° C. to 25 ml of anhydrous DMF, and the reaction stirred under argon for 30 minutes (the colour of the reaction becomes light yellow). The dipyrromethane [14] (3.4 mg, 12.5 mmol) was dissolved in anhydrous DMF (20 ml) and cooled down to 0° C., the previously prepared Vilsmeier reagent was then added dropwise. The reaction mixture was stirred under argon at 0° C. for 2 h, allowed to warm to room temperature at which point the iminium salt was hydrolysed by the addition of 1M KOH solution (until pH>10). The resulting dark brown oil was extracted with ethyl acetate (2×100 ml), the combined organic layers washed with water, dried (Na₂SO₄), filtered and concentrated. The resulting oil was purified by column chromatrography [silica gel, chloroform/ethyl acetate (8:2)] to give a brown oil (73%). ¹H NMR δ (ppm, CDCl₃): 11.16 (br s, 2H, NH); 9.42 (s, 2H, —CHO); 6.92 (dd, 2H, β-pyrrole, J₁=2.47 Hz, J₂=1.23 Hz); 6.18 (dd, 2H, β′-pyrrole, J₁=2.47 Hz, J₂=1.23 Hz); 4.17 (t, 1H, methane bridge H, J=7.92 Hz); 2.11 (m, 2H, —CH₂(CH₂)₇CH₃); 1.21 (m, 14H, alkyl chain); 0.84 (m, 3H, —CH₃).

### 1,9-Bishydroxymethyl-5nonyldipyrromethane [16]

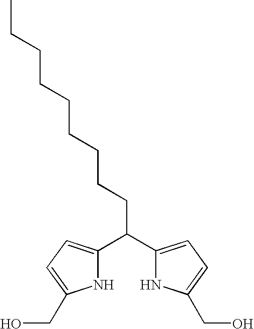

The bisformyldipyrromethane [15] (1 g, 3.05 mmol) was dissolved in a mixture of THF/methanol (3:1, 72 ml). NaBH₄ (5.76 g, 0.152 mmol) was then added in small portions and the reaction progess followed by TLC [neutral alumina oxide, DCM/5% methanol]. The reaction was stirred at room temperature and after 30 minutes quenched by addition of water. The reaction mixture was then extracted with diethyl ether (2×100 ml), the combined organic extracts washed with saturated solution of Na₂CO₃, dried (Na₂SO₄), filtered and concentrated to yield a light yellow solid (97%). ¹H NMR δ (ppm, CDCl₃): 9.82 (br s, 2H, NH); 5.91 (d, 4H, pyrrole, J=2.47 Hz); 4.27 (s, 4H, —CH₂OH); 3.84 (t, 1H, methane bridge H, J=7.42 Hz); 1.82 (m, 2H, —CH₂(CH₂)₇CH₃); 1.21 (m, 14H, alkyl chain); 0.84 (m, 3H, —CH₃).

### 5-Nonylporphyrin [17]

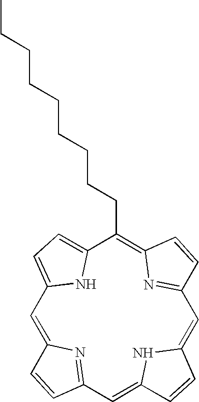

To a light protected stirred solution of the dipyrromethanediol [16] (0.17 g, 0.5 mmol) and the unsubstituted dipyrromethane (0.07 g, 0.5 mmol) in CH₃CN (200 ml), under argon, TFA was added (0.46 ml, 6 mmol) and the reaction stirred at room temperature for 1 h. DDQ (0.34 g, 1.68 mmol) was then added to oxidise the resulting pophyrinogen and the mixture stirred open to the air at room temperature for another 1 h. The resulting mixtures was neutralized by the addition of triethyl amine (0.84 ml, 6 mmol) and the solution filtered through a large pad of silica, eluting with DCM. The front running porphyrinic red band was collected and purified column chromatography [silica gel, chloroform/hexane (6:4)] to give the desired porphyrin [17] as a red solid (15%). ¹H NMR δ (ppm, CDCl₃): 10.22 (s, 2H, meso-H attached to C₁₀ and C₂₀); 10.13 (s, 1H, meso-H attached to C₁₅); 9.65 (d, 2H, pyrrole H, J=4.81 Hz); 9.43 (m, 4H, pyrrole H); 9.40 (d, 2H, pyrrole, J=4.24); 5.08 (t, 2H, —CH₂(CH₂)₇CH₃), J=8.32 Hz); 2.58 (m, 2H, —CH₂CH₂(CH₂)₆CH₃); 1.86 (m, 4H, —(CH₂)₂CH₂CH₂(CH₂)₄CH₃); 1.30 (m, 8H, CH₂)₄(CH₂)₄CH₃); 0.90 (t, 3H, —CH₂(CH₂)₇CH₃), J=6.97 Hz); −3.53 (br s, 2H, NH). ¹³C NMR δ (ppm, CDCl₃): 146.83, 144.92 (two broad doublets); 131.76, 131.54, 130.76, 128.23, 120.18, 104.14, 102.71 (rest of C at the porphyrin ring); 67.95, 38.93, 35.16, 31.88, 30.63, 29.66, 25.59, 22.65, 14.070 (alkyl chain). MS (FAB⁺) 437 (M⁺), 323. UV-Vis (CH₃Cl) λₘₐₓ (nm) 398 (ε=284000 cm⁻¹M⁻¹), 496 (ε=17000 cm⁻¹M⁻¹), 569 (ε=4700 cm⁻¹M⁻¹)

### 5-Nonane-10,15,20-triiodo-porphyrin [18]

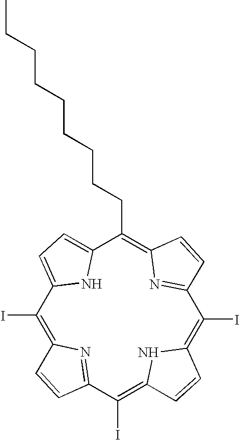

To a solution of (CF₃CO₂)₂PhI (0.26 g, 0.6 mmol) in dry CH₃Cl (40 ml) a solution of iodine (0.13 g, 0.5 mmol; in 15 ml of dry CH₃Cl) was added followed by a few drops of pyridine. The red-violet solution was stirred (shielded from light) at room temperature under argon until it turned yellow (about 15 min). This mixture was then added dropwise to a stirred solution of [17] in dry CH₃Cl (100 ml). The reaction was stirred at room temperature, under argon and in the dark. After 24 h another batch of freshly prepared iodine and (CF₃CO₂)₂PhI solution was added and the mixture stirred for a further 24 h. The reaction was monitored by TLC [silica gel, chloroform/hexane (8:2)] and on completion it was quenched by washing it with a saturated solution of sodium thiosulphate (4×100 ml). The chloroform layer was separated, dried (Na₂SO₄) and the solvent removed to yield a dark violet solid (80%). MS (FAB⁺) 815 (M⁺), 689. UV-Vis (CH₃Cl) λₘₐₓ 428, 529, 568, 611, 674 nm. Due to its limited solubility, the triiodoporphyrin [18] was not fully characterised.

### Zinc 5-nonyl-10,15,20-triiodoporphyrin [19]

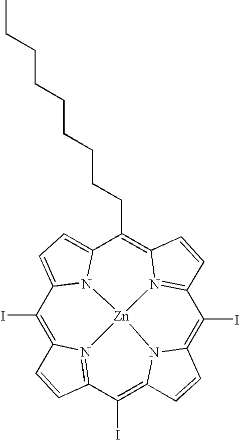

To a solution of porphyrin [18] (85 mg, 0.104 mmol) in THF/CH₃Cl/MeOH (68 ml, 25:5:4), zinc acetate (0.23 g, 1.04 mmol) was added and the mixture was refluxed for 2 hours. The reaction can be monitored spectroscopically and within 30 minutes the initial purple solution became turquoise-green. The reaction was cooled to room temperature and reduced in volume. The solid obtained was washed with methanol to remove excess zinc acetate and filtered to give the metallated porphyrin [19] as a purple solid (90%). ¹H NMR δ (ppm, THF): 9.63 (dd, 4H, pyrrole H, J=4.80 and 2.32 Hz); 9.60 (d, 2H, pyrrole H,J=4.73); 9.37 (d, 2H, pyrrole, J=4.74); 4.78 (t, 2H, —CH₂(CH₂)₇CH₃), J=7.94 Hz); 2.56 (m, 2H, —CH₂CH₂(CH₂)₆CH₃); 1.73 (m, 4H, —(CH₂)₂CH₂CH₂(CH₂)₄CH₃); 1.30 (m, 8H, CH₂)₄(CH₂)₄CH₃); 0.88 (m, 3H, —CH₂(CH₂)₇CH₃)). ¹³C NMR δ (ppm, THF): 154.21, 153.79, 152.92, 139.95, 139.64, 131.43, 124.71, 107.07, 68.22, 40.34, 36.13, 31.28, 30.68, 30.36, 25.31, 23.56, 14.47. MS (FAB⁺) 878 (M⁺), 750, 637. UV-Vis (THF) λₘₐₓ (nm) 436, 572, 621

### Zinc 5-nonyl-10,15,20-tri(2-ethynylpyridine)-porphyrin [20]

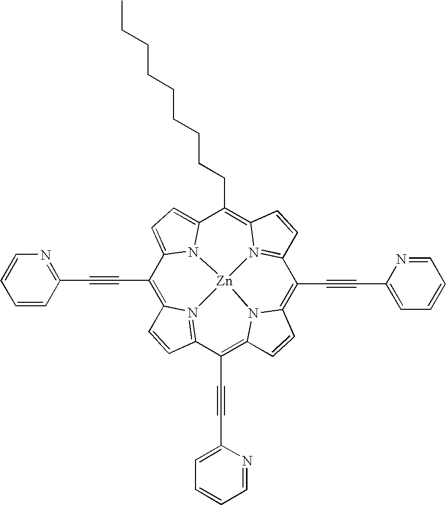

To a solution of the zinc triiodinated porphyrin [19] (60 mg, 0.07 mmol) in dry THF (10 ml), dry triethylamine (0.66 ml) was added followed by tetrakis(triphenylphosphine) palladium(0) (12 mg, 0.01 mmol) and copper iodide (5 mg, 0.025 mmol). Then 2-ethynylpyridine (70.5 mg, 0.68 mmol) was added and the reaction mixture stirred at room temperature, under argon and shielded from light. The reaction was followed by UV spectroscopy and was complete within 24 h. The resulting dark green solution was passed through a short pad of silica, eluting with THF and an intense green band was collected. The solvent was removed and the crude purified by column chromatography [silica gel, THF/hexane (6:4)] to yield the triethynylpyridine porphyrin [20] as a green solid (59%). ¹H NMR δ (ppm, THF): 9.74 (m, 6H, pyrrole H); 9.47 (d, 2H, pyrrole H, J=4.47 Hz); 8.84 (m, 3H, pyridine-H); 8.13 (d, 3H, pyridine-H, J=7.66 Hz); 7.97 (m, 3H pyridine-H); 7.45 (m,3H, pyridine-H); 4.92 (br s, 2H, —CH₂(CH₂)₇CH₃)); 2.46 (m, 2H, —CH₂CH₂(CH₂)₆CH₃); 1.77 (m, 4H, —(CH₂)₂(CH₂)₂(CH₂)₄CH₃); 1.33 (m, 8H, —(CH₂)₄(CH₂)₄CH₃); 0.88 (m, 3H, —(CH₂)₈CH₃). ¹³C NMR δ (ppm, THF): 152.31, 151.39, 150.89, 145.42, 137.02, 132.38, 131.90, 130.55, 127.95, 126.57, 123.61, 101.33, 100.23, 97.10, 96.72, 92.73, 92.47, 40.10, 36.16, 32.87, 31.33, 30.38, 25.52, 24.92, 23.58, 14.47. MS(FAB⁺): 803 (M⁺), 547. UV-Vis (THF) λₘₐₓ (nm) 465 (ε=408000 cm⁻¹M⁻¹), 606 (ε=13000 cm⁻¹M⁻¹), 666 (ε=38000 cm⁻¹M⁻¹).

### Zinc 5-nonyl-10,15,20-tri(N-methyl-2-ethynylpyridine)-porphyrin [21]

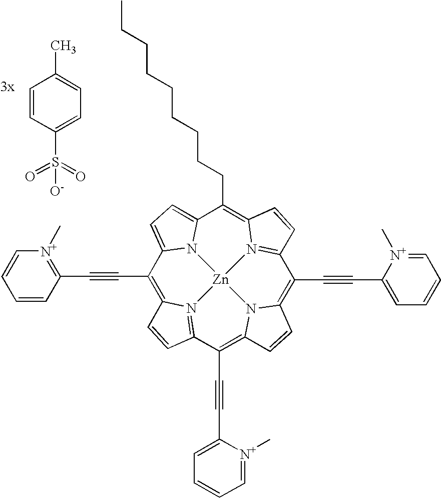

Porphyrin [20] (31 mg, 0.04 mmol) was dissolved in anhydrous DMF (1 ml) and methyl p-toluene sulphonate (60 μl, 0.4 mmol) was added. The mixture was refluxed for 4 h during which time it was monitored by UV spectroscopy. On completion the reaction was allowed to cool down to room temperature alter which it was poured into a large excess of diethylether (40 ml). The resulting crude solid was stirred in ether for several hours to complete the precipitation of the porphyrin as a dark solid which was filtered and dried (80%). MS (FAB⁺) 1365 (M⁺), 848. UV-Vis (THF) λₘₐₓ 494, 630, 686 nm.

### Zinc 5-nonyl-10,15,20-tri(m-O-tbutyldimethylsilane-ethynylphenol)-porphyrin [22]

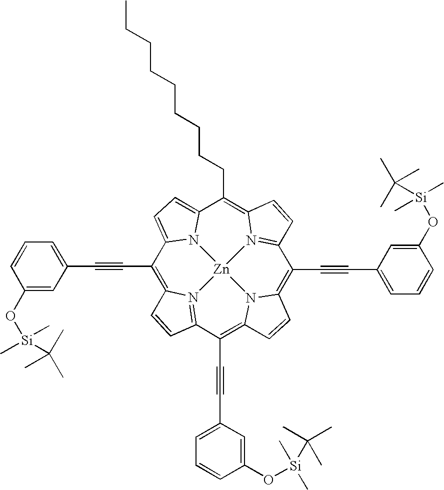

To a solution of the metallated triiodo porphyrin [19] (67 mg, 0.076 mmol) in dry THF (10 ml), dry triethylamine (0.74 ml) was added followed by tetrakis(triphenylphosphine) palladium(0) catalyst (13 mg, 0.011 mmol) and copper iodide (5.4 mg, 0.028 mmol). Then m-O-tbutyldimethylsilane-ethynylphenol (176 mg, 0.76 mmol) was added and the reaction mixture stirred at room temperature, under argon and shielded from light. The reaction was followed by UV spectroscopy and was complete within 24 h. The resulting dark green mixture was passed through a short pad of silica, eluting with THF, an intense green band was collected. The solvent was removed and the crude purified by column chromatography [silica gel, THF/hexane (6:4)] to yield the triethynyl porphyrin [22] as a green solid (77%). ¹H NMR δ (ppm, CDCl₃): 8.94 (m, 4H, pyrrole H); 8.88 (d, 2H, pyrrole H, J=4.40 Hz); 8.52 (d, 2H, pyrrole H, J=4.30 Hz); 7.67 (m, 3H, phenyl ring); 7.47 (m,6H, phenyl ring); 7.05 (m, 3H, phenyl ring); 4.00 (br s, 2H, —CH₂(CH₂)₇CH₃)); 2.28 (br s, 2H, —CH₂CH₂(CH₂)₆CH₃); 1.55 (br s, 4H, —(CH₂)₂(CH₂)₂(CH₂)₄CH₃); 1.19 (m, 8H, —(CH₂)₄(CH₂)₄CH₃); 1.18 (s, 27H, SiC(CH₃)₃); 0.87 (br s, 3H, —(CH₂)₈CH₃); 0.44 (s, 18H, Si(CH₃)₂). MS(FAB⁺): 1190 (M⁺). UV-Vis (CDCl₃) λₘₐₓ (nm) 466 (ε=307000 cm⁻¹M⁻¹), 603 (ε=10000 cm⁻¹M⁻¹), 660 (E=24000 cm⁻¹M⁻¹)

### Zinc 5-nonane-10,15,20-tri(m-ethynylphenol)-porphyrin [23]

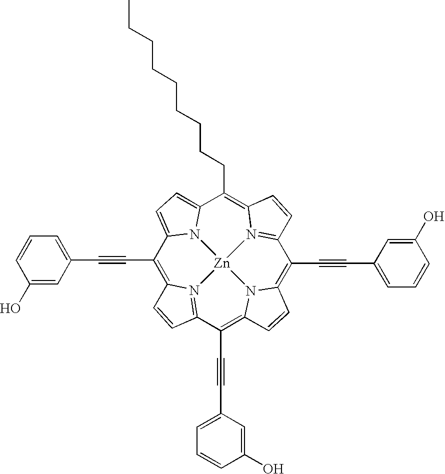

To a light protected solution of porphyrin [22] (23 mg, 0.02 mmol in dry THF (1 ml) tetrabutylammonium fluoride (100 μl, 1M solution in THF) was added dropwise and the reaction stirred for 1 h. The reaction was monitored by tlc and on completion the solvent was removed to give a sticky solid. This was triturated with water to give the porphyrin [23] as a green solid 15 mg (93%). MS (FAB⁺): 846 (M⁺). UV-Vis (MeOH) λₘₐₓ (nm) 461, 608, 668.

### 4-Iodophenyl-2,3,4,6-Tetra-O-acetyl-α-D-mannopyranoside [24]

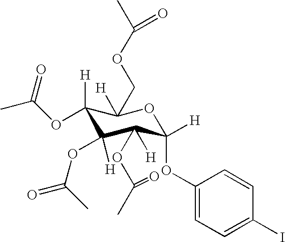

Ref. Chem Eur. J. 2000, 6(10), 1757-1762

To a solution of penta-o-acetyl-α-D-mannopyranose (1.0 g, 2.56 mmol) and 4-iodophenol (1.0 g, 4.45 mmol) in dry DCM, BF₃.OEt₂ (0.5 ml, 4.0 mmol) was added and the mixture stirred at room temperature, under argon for 18 h. The reaction was monitored by TLC [silica gel, ethylacetate/hexane (1:1)]. On completion, dichloromethane (75 ml) was added and the solution was washed with a saturated aqueous sodium carbonate solution (2×50 ml), sodium hydroxide solution (0.5N, 2×50 ml) and water (2×50 ml). The organic layer was dried over magnesium sulphate, filtered and the solvent was evaporated. Then the crude was crystallized from ether/hexane (1:3) to yield the sugar [24] as a white solid (79%). ¹H NMR δ (ppm, CDCl₃): 7.59, 6.86 (two d, 4H, AB spin syst aromatic ring, J=8.87 Hz); 5.52 (dd, 1H, J=3.56, 10.03 Hz, H₃); 5.48 (d, 1H, H₁, J=1.5 Hz); 5.42 (dd, 1H, H₂, J=3.4, 1.8 Hz); 5.35 (t, 1H, H₄, J=10.1 Hz); 4.26 (dd, 1H, H₆, J=5.44, 12.30 Hz); 4.07-4.01 (m, 2H, H₅, H₆); 2.19, 2.05, 2.03 (3s, 12H, acetate groups). ¹³C NMR δ (ppm, CDCl₃): 170.53, 169.97, 169.73 (CO); 155.36, 138.49, 118.71, 85.82 (C₆H₄); 95.67 (C₁); 69.25 (C₂); 69.19, 68.71, 65.75 (C₃, C₄, C₅); 62.00 (C₆), 20.9-20.7 (CH₃CO).

### 4-trimethylsilylethynylphenyl-2, 3,4,6-Tetra-O-acetyl-D-mannopyranoside [25]

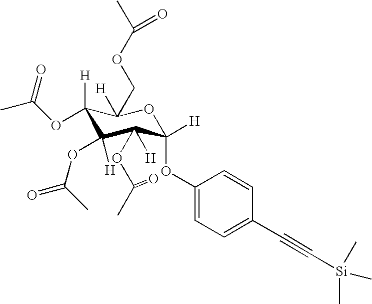

To a solution of the sugar [24] (1 g, 1.82 mmol) in dry triethylamine (7 ml), tetrakis(triphenylphosphine)palladium(0) catalyst (26 mg, 0.036 mmol) and copper iodide (35 mg, 0.18 mmol) were added. Then trimethylacetylyne (214 mg, 2.18 mmol) was added and the reaction mixture stirred at room temperature, under argon. The reaction was monitored by TLC [silica gel, ethylacetate/hexane (1:1)] and after 5 h the solvent was removed. The crude was washed with ether (3×15 ml) and the amine insoluble salt was removed. The ether layer was concentrated and the solid was purified by column chromatography [silica gel, ethylacetate/hexane (1:1)] to obtain the sugar [25] as a light brown solid (89%). ¹H NMR δ (ppm, CDCl₃): 7.41, 7.01 (two d, 4H, AB spin syst aromatic ring, J=8.79 Hz); 5.54 (d, 1H, J=3.48 Hz, H₃); 5.52 (m, 1H, H₁); 5.42 (m, 1H, H₂); 5.35 (t, 1H, H₄, J=9.97 Hz); 4.27 (dd, 1H, H₆, J=5.94, 12.78 Hz); 4.06-4.03 (m, 2H, H₅, H₆); 2.20, 2.05, 2.03 (3s, 12H, acetate groups); 0.23 (s, 9H, Si(CH₃)₃). ¹³C NMR δ (ppm, CDCl₃): 170.55, 169.98, 169.74 (CO); 155.47, 133.49, 116.24, 104.38 (C₆H₄); 95.56, 93.51, 69.22, 68.73, 65.79, 62.02 (sugar C), 20.9-20.7 (CH₃CO); −0.03 (Si(CH₃)₃). MS (FAB⁺): 521 (M⁺), 331.

### 4-ethynylphenyl-2, 3,4,6-Tetra-O-acetyl-D-mannopyranoside [26]

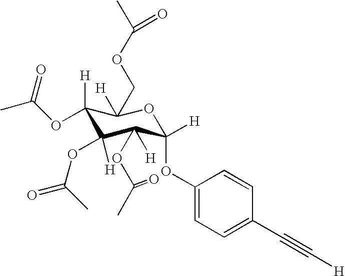

Sugar [25] (0.72 g, 1.38) was dissolved in dry THF (30 ml) and tetrabutylammonium fluoride (2 ml, 1M solution in THF) was added dropewise. The reaction was allowed to procedd at room temperature, under argon, for 1 h. The reaction mixture was concentrated and the brown oily crude was purified by column chromatography [silica gel, dichloromethane/ethylacetate (8:2)] to yield the sugar [26] as a light brown solid. ¹H NMR δ (ppm, CDCl₃): 7.44, 7.03 (two d, 4H, AB spin syst aromatic ring, J=8.77 Hz); 5.53 (m, 2H, H₃, H₁); 5.43 (m, 1H, H₂); 5.35 (t, 1H, H₄, J=9.98 Hz); 4.27 (dd, 1H, H₆, J=5.75, 12.55 Hz); 4.07-4.02 (m, 2H, H₅, H₆); 3.03 (s, 1H, —C≡C—H); 2.20, 2.05, 2.03 (3s, 12H, acetate groups). ¹³C NMR δ (ppm, CDCl₃): 170.55, 169.98, 169.74 (CO); 155.68, 138.65, 116.35, 82.98 (C₆H₄); 95.54 (C₁); 69.25 (C₂); 69.19, 68.72, 65.77 (C₃, C₄, C₅); 62.01 (C₆), 31.59 (—C≡C—H); 20.9-20.7 (CH₃CO).

### Zinc5-nonyl-10,15,20-tri[p-ethynylphenyl-2,3,4,6-Tetra-O-acetyl-D-mannopyranoside]-porphyrin [27]

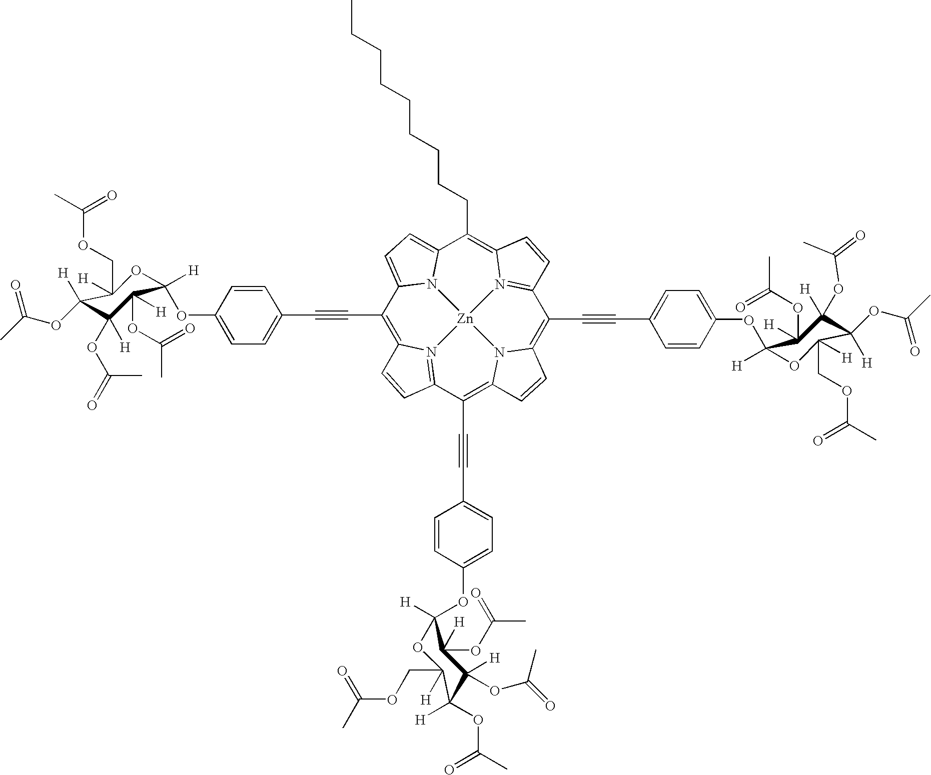

To a solution of the metallated triiodo porphyrin [19] (59 mg, 0.067 mmol) in dry THF (10 ml), dry triethylamine (0.66 ml) was added followed by tetrakis(triphenylphosphine) palladium(0) catalyst (12 mg, 0.01 mmol) and copper iodide (4.7 mg, 0.025 mmol). Then the sugar [13] (0.3 g, 0.67 mmol) was added and the reaction mixture stirred at room temperature, under argon and shielded from light. The reaction was followed by UV spectroscopy and was complete within 24 h. The resulting dark green mixture was passed through a short pad of silica, eluting with THF an intense green band. The solvent was removed and the crude purified by column chromatography [first column with silica gel, THF/hexane (6:4) and a 2nd column with silica gel, dichloromethane/ethylacetate (8:2) increasing the polarity to (5:5)] to yield the triacetylated sugar porphyrin [27] as a green solid (55%). ¹H NMR δ (ppm, CDCl₃): 9.24 (m, 4H, pyrrole-H); 9.12 (m, 2H, pyrrole-H); 8.86 (m, 2H, pyrrole-H); 7.47, 7.05 (two d, 4H, AB spin syst aromatic ring, J=8.77 Hz); 5.68 (m, 6H, sugar-H); 5.46 (m, 6H, sugar-H); 4.36 (m, 5H, 3H from sugar and —CH₂(CH₂)₇CH₃); 4.21 (m, 6H, sugar-H); 2.26 (m, 2H, —CH₂CH₂(CH₂)₆CH₃); 2.24, 2.09, 2.05 (3s, 36H, acetate groups); 1.59 (m, 4H, —(CH₂)₂(CH₂)₂(CH₂)₄CH₃); 1.42 (m, 8H, —(CH₂)₄(CH₂)₄CH₃); 0.87 (m, 3H, —(CH₂)₈CH₃)). MS (FAB⁺) 1838 (M⁺), 1507. UV-Vis (MeOH) λₘₐₓ (nm) 468 (ε=118000 cm⁻¹M⁻¹), 606 (ε=3900 cm⁻¹M⁻¹), 664 (ε=10400 cm⁻¹M⁻¹).

### Zinc 5-nonyl-10,15,20-tri[p-ethynylphenol-O-(D-mannopyranose)]-porphyrin [28]

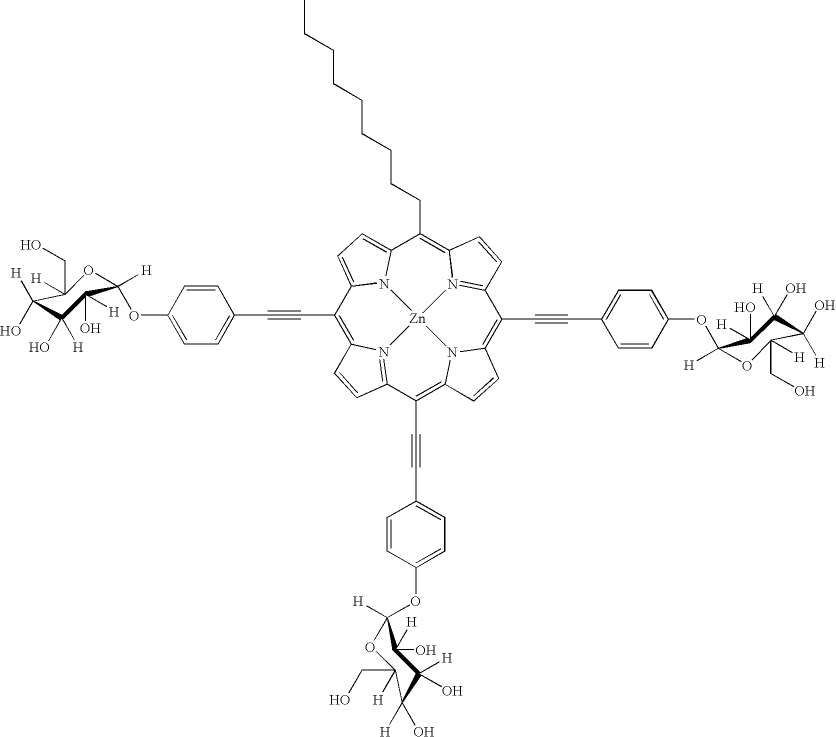

Ref. J. of Biomed. Optic. July 1999, 4(3), 298

To a light protected solution of the sugar porphyrin [27] (29 mg, 0.016 mmol) in dry methanol (10 ml), sodium methoxide (200 μl, 1M solution in dry methanol) was added and the mixtured was stirred for 1 h, under argon, at room temperature. The solvent was removed and the solid washed with water to yield the porphyrin [28] as a green solid (74%) MS (FAB⁺) 1005 (M-327), 678 (M-654), 351 (M-981). UV-Vis (MeOH) λₘₐₓ (nm) 463, 612, 671.

### 5-Pentane porphyrin [29]

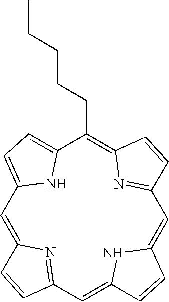

Obtained as a side product (3-4% yield) from the synthesis of 5-bromopentane porphyrin ¹H NMR δ (ppm, CDCl₃): 10.22 (s, 2H, meso-H attached to C₁₀ and C₂₀); 10.12 (s, 1H, meso-H attached to C₁₅); 9.64 (d, 2H, pyrrole H, J=4.65 Hz); 9.43 (d, 4H, pyrrole H, J=5.16 Hz); 9.39 (d, 2H, pyrrole, J=4.42 Hz); 5.07 (t, 2H, —CH₂(CH₂)₃CH₃), J=8.13 Hz); 2.60 (m, 2H, —CH₂CH₂(CH₂)₂CH₃); 1.83 (m, 2H, —(CH₂)₂CH₂CH₂CH₃); 1.58 (m, 2H, CH₂)₃CH₂CH₃); 1.01 (t, 3H, —(CH₂)₄CH₃), J=7.30 Hz); −3.59 (br s, 2H, NH). ¹³C NMR δ (ppm, CDCl₃): 146.69, 144.72 (two broad doublets); 131.69, 130.69, 128.15, 127.50, 120.08, 104.81, 102.65 (rest of C at the porphyrin ring); 38.24, 35.03, 32.71, 22.72, 14.90 (alkyl chain). MS (FAB⁺) 380 (M⁺), 323.

### 5-Pentane-10,15,20-triiodo-porphyrin [30]

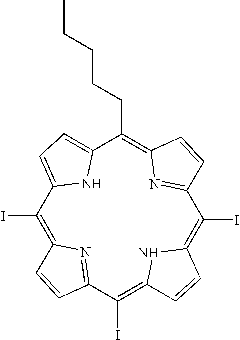

To a solution of (CF₃CO₂)₂PhI (0.24 g, 0.55 mmol) in dry CH₃Cl (40 ml) a solution of iodine (118 mg, 0.46 mmol; in 15 ml of dry CH₃Cl) was added followed by a few drops of pyridine. The red-violet solution was stirred (shielded from light) at room temperature under argon until it became yellow (about 15 min). This mixture was then added dropwise to a stirred solution of 5-pentane-porphyrin [29] in dry CH₃Cl (100 ml). The reaction was stirred at room temperature, under argon and in the dark. After 24 h another batch of the iodine and (CF₃CO₂)₂PhI solution was added and the mixture was stirred for a further 24 h. The reaction was monitored by TLC [silica gel, chloroform/hexane (8:2)] and on completion it was quenched by washing it with a saturated solution of sodium thiosulphate (4×100 ml). The chloroform layer was separated, dried (Na₂SO₄) and the solvent removed to yield a dark violet solid (93%). MS (FAB⁺) 759 (M⁺). UV-Vis (THF) λₘₐₓ 430, 531, 670, 673. Due to its limited solubility, the triiodoporphyrin [30] was not fully characterised.

### Zinc 5-pentane-10,15,20-triiodo-porphyrin [31]

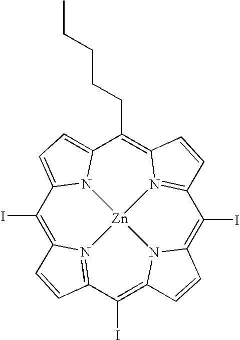

To a solution of porphyrin [30] (162 mg, 0.214 mmol) in THF/CH₃CUMeOH (80 ml, 25:5:4), zinc acetate (0.47 g, 2.14 mmol) was added and the mixture was refluxed for 2 hours. The reaction can be monitores with UV and within 30 min the initially purple solution became green. Then, the reaction was cooled to room temperature and the voume was reduced. The solid obtained was washed with methanol to remove the excess of zinc acetate and filtered to give the metallated porphyrin [31] as a purple solid (90%). UV-Vis (THF) λₘₐₓ (nm) 435, 573, 622.

### Zinc 5-nonane-10,15,20-tri(2-ethynylpyridine)-porphyrin [32]

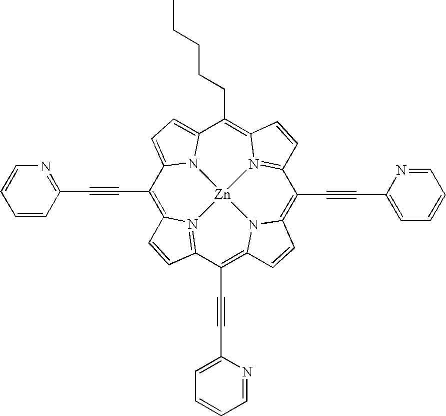

To a solution of the metallated triiodinated porphyrin [31] (80 mg, 0.097 mmol) in dry THF (15 ml), dry triethylamine (11.0 ml) was added followed by tetrakis(triphenylphosphine)palladium(0) catalyst (17 mg, 0.015 mmol) and copper iodide (7 mg, 0.036 mmol). Then 2-ethynylpyridine (100 mg, 0.97 mmol) was added and the reaction mixture stirred at room temperature, under argon and shielded from light. The reaction was followed by UV spectroscopy and was complete within 24 h. The resulting dark green mixture was passed through a short pad of silica, eluting with THF an intense green band. The solvent was removed and the crude product purified by column chromatography [silica gel, THF/hexane (6:4)] to yield the triacetylated porphyrin [32] as a green solid (58%). ¹H NMR δ (ppm, dmso): 9.59 (m, 6H, pyrrole H); 9.48 (m, 2H, pyrrole H); 8.87 (m, 3H, pyridine-H); 8.25 (m, 3H, pyridine-H); 8.07 (m, 3H pyridine-H); 7.58 (m,3H, pyridine-H); 4.78 (br s, 2H, —CH₂(CH₂)₃CH₃)); 2.31 (m, 2H, —CH₂CH₂(CH₂)₂CH₃); 1.71 (m, 2H, —(CH₂)₂CH₂CH₂CH₃); 1.47 (m, 2H, CH₂)₃CH₂CH₃); 0.92 (t, 3H, —(CH₂)₄CH₃), J=7.29 Hz). ¹³C NMR δ (ppm, dmso): 151.53, 150.57, 149.67, 143.17, 137.06, 131.55, 131.06, 130.75, 127.58, 126.28, 123.61, 99.74, 98.51, 96.07, 95.65, 91.52, 91.26, 38.73, 34.70, 31.95, 22.21, 14.10. MS(FAB⁺): 746 (M⁺), 307. UV-Vis (THF) λₘₐₓ 465, 607, 666 nm.

### Zinc 5-pentane-10,15,20-tri(N′-methyl-2-ethynylpyridine)-porphyrin [33]

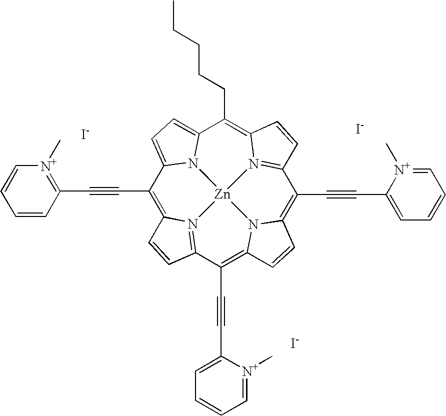

To a light protected solution of porphyrin [32] (20 mg, 0.027 mmol) in DMF (1 ml), iodomethane (0.5 ml) was added. The reaction was stirred over 4 h at room temperature, under argon. Then the solvent was removed to yield porphyrin [33] as a green solid (85%). UV-Vis (MeOH) λₘₐₓ 496, 627, 687 nm.

Determination of Singlet Oxygen Quantum Yields (φ)

Air equilibrated solutions of the sensitisers were optically matched at the laser excitation wavelength along with that of the reference standard whose singlet oxygen quantum yield is known. Singlet oxygen generation is detected by its phosphorescence at 1270 nm following laser excitation. At each laser intensity the recorded phosphorescence trace was obtained by signal averaging 10 single shots. A linear regression between the signal amplitude and the laser intensity is carried out with the aim of calculating the slope of the straight line and since the gradient is proportional to the singlet oxygen quantum yield, by comparison with the gradient obtained for the standard, the singlet oxygen quantum yield for the sample can calculated using:
φ=φ_{standard}×(slopeₛₐₘₚₗₑ/slope_{standard})×(absorptionₛₐₘₚₗₑ/absorption_{standard})

This is a comparative method where the value obtained for φ is relative to that of a known standard. The quality of the results obtained are heavily dependent on the quality of the standard, which should be freshly prepared before measurements.

Results (see FIG. 6)

| Q | Power | Cpd [11] | Power | Ref 1 |
|---|---|---|---|---|
| 300 | 3.98 | 4 | 3.7 | 2 |
| 290 | 4.8 | 4.9 | 4.7 | 2.2 |
| 280 | 5.96 | 5.5 | 5.72 | 3.1 |
| 270 | 6.9 | 6.3 | 6.9 | 2.9 |
| 260 | 8.16 | 7.3 | 7.88 | 3.7 |
| 250 | 9.4 | 7.9 | 9.1 | 3.9 |
| 240 | 10.9 | 9.1 | 10.8 | 4.4 |
| 230 | 11.9 | 10 | 11.7 | 4.9 |
| 220 | 13.7 | 10.6 | 13.2 | 5.4 |
| 210 | 14.6 | 11.2 | 13.8 | 5.6 |
| 200 | 15.4 | 12.1 | 15.1 | 6.1 |
| 190 | 15.5 | 12.5 | 15.6 | 6.1 |
| 180 | 15.9 | 12.4 | 16 | 6.1 |

Linear regression for compound [11], zinc-5-[5-(2′-(trimethylsilyl)ethyl-4-hydroxybenzoate)pentane]-10,15,20-tri-2-ethynylpyridine-porphyrin, in toluene (FIG. 6A):

y=0.6899x+1.4783

R2=0.9947

Linear regression for reference (chlorophyll A) in toluene (FIG. 6C):

y=0.3444x+0.7835

R2=0.9867

| nm | abs | correct abs | |
|---|---|---|---|
| 570 | 0.026582 | 0.026582 | Reference |
| 570 | 0.045191 | 0.039399 | Compound [11] |

The singlet oxygen quantium yield for compound [11] in toluene was 0.810918. The singlet oxygen quantium yield for reference (chlorophyll A) in toluene was 0.6.

Synthesis and Utility of scFv-4 Helix Bundle Fusion Protein Carrying PS Drug Molecules

A chosen, well characterised scFv is PCR amplified and cloned as an Nco I/Not I fragment into the bacterial expression vector pET20b (Novagen) to create pETscFv. A DNA cassette containing a 4 helix bundle (e.g. a derivative of the bacterial protein ‘rop’) is PCR amplified and cloned into the Not I site of pETscFv to create pETscFv4HB (FIG. 3). Appropriate DNA primers are used introduce cysteine residues at optimal positions in the helix bundle and to replace any cysteine residues in the scFv (with residues which do not significantly alter the binding characteristics of the scFv, such as serine, alanine and glycine). The resulting construct is called pETscFv4HBcys

The vector pETscFv4HBcys is transformed into E. coli BL21(DE3) (Novagen) by the calcium chloride method and plated onto 2TY agar plates containing 100 μg/ml ampicillin [Sambrook et al. (1989). DNA Cloning. A Laboratory Manual. Cold Spring Harbor]. Single colony transformants are picked and re-streaked onto fresh 2TY Agar plates containing amplicillin.

A single colony is picked and grown in 5 ml of 2TY media containing 100 μg/ml ampicillin at 30° C., in a shaking incubator (250 rpm) for 8-16 hr. This culture is then used to inoculate a culture of 500 ml 2TY media containing 100 μg/ml ampicillin and grown under similar conditions for a further 3-16 hr.

The culture supernatant is harvested and concentrated using an Amicon ultrafiltration stirred cell with a 30 KDa cut-off membrane to a final volume of 10 ml. Alternatively, the bacterial periplasm can be prepared using the sucrose osmotic shock method [Deonarain M P & Epenetos A A (1998) Br. J. Cancer. 77, 537-46. Design, characterization and anti-tumour cytotoxicity of a panel of recombinant, mammalian ribonuclease-based immunotoxins] in a volume of 10 ml.

The concentrated supernatant or periplasmic extract is dialysed for 16 hr against 5 L of phosphate-buffered saline (PBS) containing 0.5 M NaCl and 2 mM MgCl₂. This is then applied to a copper (II) or nickel (II)-charged chelating sepharose column (Amersham-Pharmacia Biotech) and purified by immobilised metal affinity chromatography (IMAC) for example as described in Deonarain et al [Deonarain M P & Epenetos A A (1998) Br. J. Cancer. 77, 537-46. Design, characterization and anti-tumour cytotoxicity of a panel of recombinant, mammalian ribonuclease-based immunotoxins]. The recombinant fusion protein should elute in an imidazole gradient at between 40 and 150 mM imidazole. The eluted fusion protein is further purified by gel filtration on a superdex-200 column (Amersham-Pharmacia Biotech) equilibrated in PBS. FIG. 4 shows shows data for the expression and purification of the resulting fusion protein, scFv-4-helix bundle-cys.

Preparation of pETscFv4HBLys

A scFv-4 helix bundle was prepared in accordance with the methodology described above, except that appropriate primers were used to introduce lysine residues at optimal positions in the helix bundle. The resulting construct is called pETscFv4HBLys. An scFv which targets CEA (carcinoembryonic antigen) was used.

Coupling of Porphyrin to scFv4-Helix Bundle-Lys

The N-hydroxysuccinimide (NHS) ester of the photosensitiser porphyrin, was prepared by reacting 1.5 equivalents of dicyclohexylcarbodiimide and 1.5 equivalents of NHS with one equivalent of porphyrin in dry dimethyl sulphoxide (DMSO). The reaction was carried out under an inert gas (eg argon) and in the dark at room temperature and was complete in 2 hours, (tic: silica gel 3% methanol in chloroform). A similar procedure can be used to prepare the active ester of any carboxyl containing photosensitiser.

N-ethylmorpholine (1 μl), DMSO (10 ml) and the scFv4-helix bundle (100 μg in approx. 1 ml of PBS buffer) were stirred together in the dark and under nitrogen at room temperature. To this solution was added the DMSO solution containing the photosensitiser-NHS ester. The solution was stirred at room temperature in the dark for 12 hours to synthesise the bundle photosensitiser porphyrin conjugate. The conjugate was then dialysed against 2×5 L of PBS. All procedures were carried out in the dark.

The number of porphyrins attached to the 4-helix bundle fusion protein is determined using electrospray mass spectrometry, compared to the 4-helix bundle alone. To confirm the position of attachment on the 4-helix bundle, the protein will be fragmented by trypsin digestion and the resulting peptides analysed by mass spectrometry.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in the relevant art are intended to fall within the scope of the following claims.

1. A compound of formula I, or salt thereof, 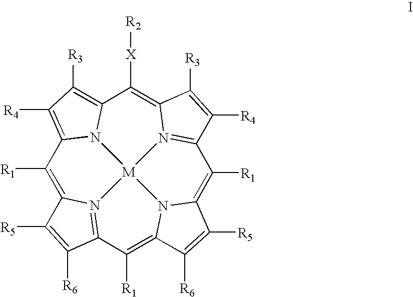
wherein
each R₁ is independently 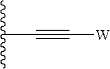
wherein W is an aryl, alkyl or heteroaryl group, at least some of which may be optionally substituted by one or more of:
OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, or a sugar derivative, Q;
R₂ is H, a halogen, an isothiocyanate group, a haloacetamide, maleimide, Y-aryl or Y-heteroaryl, where Y is O, S, NH, C(O) or CO₂, and where said aryl or heteroaryl group may be optionally substituted by one or more of:
OH, halogen, an isothiocyanate group, a haloacetamide, maleimide, COOH, NO₂, NH₂, alkyl, haloalkyl, alkoxy, (CO)_{n′}(O)_{m′}Z′, a polyethylene glycol group, an alkyl sulfonate group, an alkyl-COOH group, a substituted or unsubstituted benzyl group, or a sugar derivative, Q′;
Z and Z′ are each independently silicon-containing protecting groups and m, m′, n and n′ are each independently 0 or 1;
X is a C₁₋₂₀ alkylene group, optionally substituted by one or more substituents selected from halogen, NO₂, CN, OH, OMe, NH₂, CF₃, COOH and CONH₂;
each R₃, R₄, R₅ and R₆ is independently H, alkyl, alkoxy, halogen or OH; and
M is 2H or a metal.

2. A compound according to claim 1 wherein X is a C₁₋₁₀ alkylene group.

3. A compound according to claim 2 wherein X is a C₅₋₁₀ alkylene group.

4. A compound according to claim 1, wherein R₂ is H, halo or is selected from the following: 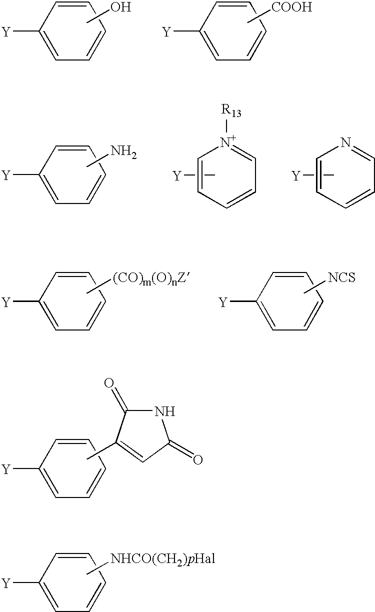
wherein R₁₃ is an alkyl group, an alkyl sulfonate group, an alkyl-COOH group or a substituted or unsubstituted benzyl group, and p is an integer from 1 to 10.

5. A compound according to claim 1 wherein R₂ is H, a halogen, or Y-aryl.

6. A compound according to claim 1, wherein R₂ is selected from the following:

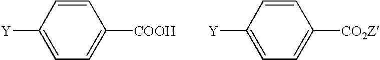

7. A compound according to claim 1, wherein Y is O.

8. A compound according to claim 1, wherein said silicon-containing protecting group, Z′, is (CH₂)_{q′}Si(R₇)(R₈)(R₉), wherein R₇, R₈ and R₉ are each independently hydrocarbyl groups and q′ is 0, 1, 2, 3, 4 or 5.

9. A compound according to claim 8 wherein R₇, R₈ and R₉ are each independently alkyl groups.

10. A compound according to claim 8 wherein Z′ is CH₂CH₂SiMe₃.

11. A compound according to claim 1 wherein R₂ is selected from the following:

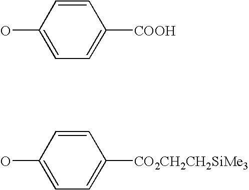

12. A compound according to claim 1 wherein R₃, R₄, R₅ and R₆ are H.

13. A compound according to claim 1 wherein each R₁ is independently 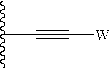
wherein W is an aryl or heteroaryl group, at least some of which may be optionally substituted by one or more of:
OH, halogen, an isothiocyanate group, a haloacetamide, maleimide, COOH, NO₂, NH₂, alkyl, haloalkyl, alkoxy, (CO)ₙ(O)ₘZ, a polyethylene glycol group, an alkyl sulfonate group, an alkyl-COOH group, a substituted or unsubstituted benzyl group, and a sugar derivative.

14. A compound according to claim 1 wherein W is an optionally substituted phenyl group.

15. A compound according to claim 1 wherein W is an optionally substituted pyridyl group.

16. A compound according to claim 1 wherein W is selected from the following: 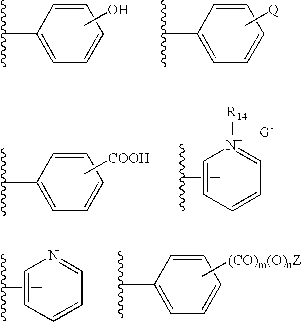
wherein R¹⁴ is an alkyl group, an alkyl sulfonate group, an alkyl-COOH group or a substituted or unsubstituted benzyl group, and G⁻ is a counter ion.

17. A compound according to claim 16 wherein Q is D-mannopyranoside or a derivative thereof.

18. A compound according to claim 16 wherein W is selected from the following:

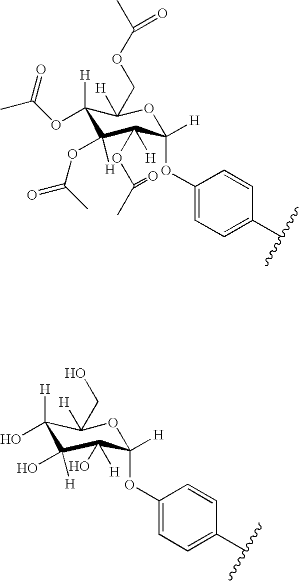

19. A compound according to claim 1 wherein said silicon-containing protecting group, Z, is (CH₂)_{q}Si(R₁₀)(R₁₁)(R₁₂), wherein R₁₀, R₁₁ and R₁₂ are each independently hydrocarbyl groups, and q is 0, 1, 2, 3, 4 or 5.

20. A compound according to claim 19 wherein R₁₀, R₁₁ and R₁₂ are each independently alkyl groups.

21. A compound according to claim 1 where W is selected from the following: 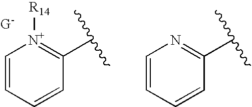
and G⁻ is halide or p-toluene sulfonate.

22. A compound according to claim 1 wherein M is selected from 2H, Ni, Pb, V, Pd, Co, Nb, Al, Sn, Zn, Cu, Mg, Ca, In, Ga, Fe, Eu, Lu, Pt, Ru, Mn and Ge.

23. A method for preparing the compound of claim 1, comprising:
reacting a compound of formula II with a dipyrrole of formula III to form a compound of formula Ib, in which R₁ is H, and X, R₂ are as defined in claim 1,

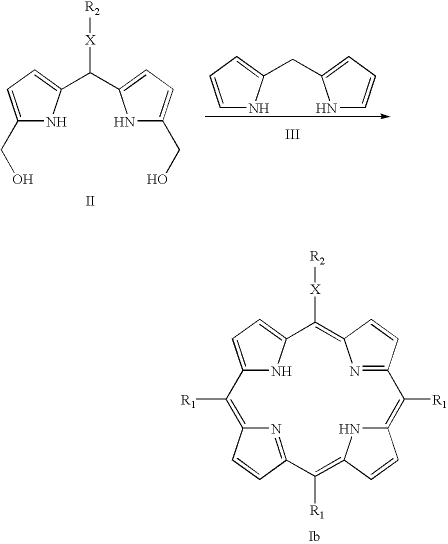

24. The method of claim 23, further comprising the step of converting said compound of formula Ib to a compound of formula Ic, in which R₁ is halogen, and X, R₂ are as defined in claim 1.

25. The method of claim 24, further comprising reacting the compound of formula Ic with 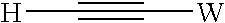
to form a compound of formula Id wherein R₁ is 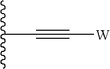
and X, R₂ and W are defined as in claim 1.


SEQUENCE LISTING (14 sequences)

SEQ ID NO: 1 - protein, 7 aa, Artificial Sequence
  Synthetically generated peptide
KKKKRPR

SEQ ID NO: 2 - protein, 17 aa, Artificial Sequence
  Synthetically generated peptide
KRPMNAFIVWSRDQRRK

SEQ ID NO: 3 - protein, 55 aa, Artificial Sequence
  Synthetically generated peptide
MLVHLFRVGIRGGPFPGRLLPPLRFQTFSAVRYSDGYRSSSLLRAVAHLPSQLWA

SEQ ID NO: 4 - protein, 4 aa, Artificial Sequence
  Synthetically generated peptide
KCPL

SEQ ID NO: 5 - protein, 4 aa, Artificial Sequence
  Synthetically generated peptide
KDEL

SEQ ID NO: 6 - protein, 23 aa, Artificial Sequence
  Synthetically generated peptide
GLFGAIAGFIENGWEGMIDGWYG

SEQ ID NO: 7 - protein, 18 aa, Artificial Sequence
  Synthetically generated peptide
GIEDLISEVAQGALTLVP

SEQ ID NO: 8 - protein, 30 aa, Artificial Sequence
  Synthetically generated peptide
ACYCRIPACIAGERRYGTCIYQGRLWAFCC

SEQ ID NO: 9 - protein, 29 aa, Artificial Sequence
  Synthetically generated peptide
FFGAVIGTIALGVATSAQITAGIALAEAR

SEQ ID NO: 10 - protein, 14 aa, Artificial Sequence
  Synthetically generated peptide
KKGKKGRKGGRKGK

SEQ ID NO: 11 - protein, 15 aa, Artificial Sequence
  Synthetically generated peptide
EGGDDEDGDEGGDED

SEQ ID NO: 12 - protein, 14 aa, Artificial Sequence
  Synthetically generated peptide
YGSGYGYSYGYYGS

SEQ ID NO: 13 - protein, 14 aa, Artificial Sequence
  Synthetically generated peptide
CGCGCGCSCGGGGC

SEQ ID NO: 14 - protein, 15 aa, Artificial Sequence
  Synthetically generated peptide
AGGLLVGVLALLGGA